(19) United States
(12) Patent Application Publication
(10) Pub. No.: US 2026/0306507 A1
(43) Pub. Date: Oct. 8, 2026

(54) IDENTIFICATION OF MECHANOSENSING REGULATORS IN PLASMACYTOID DENDRITIC CELLS

(71) Applicant: NEW YORK SOCIETY FOR THE RELIEF OF THERUPTURED AND CRIPPLED, MAINTAINING THE HOSPITAL SPECIAL --, New York, NY (US)

(72) Inventors: Vidyanath Chaudhary, New York, NY (US); Franck J. Barrat, Eastchester, NY (US)

(21) Appl. No.: 19/480,521

(22) PCT Filed: May 2, 2024
(86) PCT No.: PCT/US2024/027493

§ 371 Date: Oct. 31, 2025

Related U.S. Application Data
(60) Provisional application No. 63/463,537, filed on May 2, 2023.

(51) Int. Cl. A61K 31/554; A61K 31/225; A61P 17/00; A61P 29/00

(52) U.S. Cl. CPC A61K 31/554; A61K 31/225; A61P 17/00; A61P 29/00

(57) ABSTRACT

The present application is directed to the identification of mechanosensing regulators in plasmacytoid Dendritic Cells.

### CROSS-REFERENCE TO RELATED APPLICATIONS

The present application claims the benefit of U.S. Provisional Application No. 63/463,537, filed May 2, 2023, which is incorporated herein by reference in its entirety.

### SEQUENCE LISTING

This application contains a Sequence Listing that has been submitted electronically as an XML file named “27601-0087WO1_ST26_SL.XML.” The XML file, created on May 2, 2024, is 32,551 bytes in size. The material in the XML file is hereby incorporated by reference in its entirety.

### FIELD OF THE INVENTION

The present application is directed to the identification of mechanosensing regulators in plasmacytoid Dendritic Cells.

### BACKGROUND OF THE INVENTION

Plasmacytoid dendritic cells (pDCs) infiltrate the skin, chronically produce type I interferon (IFN-I) and promote skin lesions and fibrosis in autoimmune patients. However what controls their activation in the skin is unknown. The studies disclosed herein show that increased stiffness characteristic of the skin microenvironment inhibits the production of IFN-I by TLR-activated pDCs, contributing to resolution of local immune activation. Mechanistically, mechanosensing activates the transcription factor NRF2, which, by triggering the pentose phosphate pathway, rewires glycolysis and reduces pyruvate levels, a product necessary for pDC responses. Modulating NRF2 expression in skin-infiltrating pDCs controlled the IFN-I response, leading to either resolution or the sustained activation of pathologic IFN-I pathway in the skin. However, the studies disclosed herein show that CXCL4, a pro-fibrotic chemokine that is elevated in fibrotic skin, was able to overcome stiffness-mediated IFN-I inhibition, allowing chronic IFN-I responses by pDCs.

Hence, the data disclosed herein identifies a novel regulatory mechanism exerted by the skin microenvironment and identifies points of dysregulation of this mechanism in patients with skin inflammation. Most of the autoimmune diseases have very few drugs approved. The newly identified involvement of the pathways in blocking the pDCs and macrophages activation can lead to the discovery of several new targets for drugs development.

### SUMMARY

Provided herein is a method of inhibiting pDC activity, said method comprising activation of NRF2.

Some embodiments provide a method of inhibiting pDC activity, said method comprising inducing the Pentose Phosphate pathway.

Some embodiments provide a method of inhibiting pDC activity, said method comprising inducing or activating G6PD.

Some embodiments provide a method of inhibiting pDC activity, said method comprising inducing or activating TKT.

In some embodiments, the pDCs are in skin tissue.

Also provided herein is a method of treating a disease or disorder associated with inhibition of pDC activity, said method comprising activation of NRF2.

Some embodiments provide a method of treating a disease or disorder associated with inhibition of pDC activity, said method comprising inducing the Pentose Phosphate pathway.

Some embodiments provide a method of treating a disease or disorder associated with inhibition of pDC activity, said method comprising inducing or activating G6PD.

Some embodiments provide a method of treating a disease or disorder associated with inhibition of pDC activity, said method comprising inducing or activating TKT.

In some embodiments, the disease or disorder is an autoimmune disorder or a fibrotic disorder.

In some embodiments, the disease or disorder is a skin disease or disorder.

### BRIEF DESCRIPTION OF THE FIGURES

FIG. 1A shows that increasing stiffness or physical pressure inhibits interferon via integrin-β1 as noted in Example 1.

FIG. 1B shows that increasing stiffness or physical pressure inhibits interferon via integrin-β1 as noted in Example 1.

FIG. 1C shows that increasing stiffness or physical pressure inhibits interferon via integrin-β1 as noted in Example 1.

FIG. 1D shows that increasing stiffness or physical pressure inhibits interferon via integrin-β1 as noted in Example 1.

FIG. 1E shows that increasing stiffness or physical pressure inhibits interferon via integrin-β1 as noted in Example 1.

FIG. 1F shows that increasing stiffness or physical pressure inhibits interferon via integrin-β1 as noted in Example 1.

FIG. 1G shows that increasing stiffness or physical pressure inhibits interferon via integrin-β1 as noted in Example 1.

FIG. 2A shows that the hippo pathway components are absent in human pDCs as noted in Example 2.

FIG. 2B shows that the hippo pathway components are absent in human pDCs as noted in Example 2.

FIG. 2C shows that the hippo pathway components are absent in human pDCs as noted in Example 2.

FIG. 3A shows that the stiffness of skin inhibits interferon via activation of NRF2 transcriptional factor as noted in Example 3.

FIG. 3B shows that the stiffness of skin inhibits interferon via activation of NRF2 transcriptional factor as noted in Example 3.

FIG. 3C shows that the stiffness of skin inhibits interferon via activation of NRF2 transcriptional factor as noted in Example 3.

FIG. 3D shows that the stiffness of skin inhibits interferon via activation of NRF2 transcriptional factor as noted in Example 3.

FIG. 3E shows that the stiffness of skin inhibits interferon via activation of NRF2 transcriptional factor as noted in Example 3.

FIG. 3F shows that the stiffness of skin inhibits interferon via activation of NRF2 transcriptional factor as noted in Example 3.

FIG. 3G shows that the stiffness of skin inhibits interferon via activation of NRF2 transcriptional factor as noted in Example 3.

FIG. 3H shows that the stiffness of skin inhibits interferon via activation of NRF2 transcriptional factor as noted in Example 3.

FIG. 3I shows that the stiffness of skin inhibits interferon via activation of NRF2 transcriptional factor as noted in Example 3.

FIG. 4A show that NRF2 activate glucose 6-phosphate dehydrogenases by binding to its promoter and inhibits interferon as noted in Example 4.

FIG. 4B shows that NRF2 activate glucose 6-phosphate dehydrogenases by binding to its promoter and inhibits interferon as noted in Example 4.

FIG. 4C shows that NRF2 activate glucose 6-phosphate dehydrogenases by binding to its promoter and inhibits interferon as noted in Example 4.

FIG. 4D shows that NRF2 activate glucose 6-phosphate dehydrogenases by binding to its promoter and inhibits interferon as noted in Example 4.

FIG. 4E shows that NRF2 activate glucose 6-phosphate dehydrogenases by binding to its promoter and inhibits interferon as noted in Example 4.

FIG. 4F shows that NRF2 activate glucose 6-phosphate dehydrogenases by binding to its promoter and inhibits interferon as noted in Example 4.

FIG. 4G shows that NRF2 activate glucose 6-phosphate dehydrogenases by binding to its promoter and inhibits interferon as noted in Example 4.

FIG. 4H shows that NRF2 activate glucose 6-phosphate dehydrogenases by binding to its promoter and inhibits interferon as noted in Example 4.

FIG. 4I shows that NRF2 activate glucose 6-phosphate dehydrogenases by binding to its promoter and inhibits interferon as noted in Example 4.

FIG. 5A shows that skin stiffness induces NRF2 expression in SSc patients, and CXCL4 overcomes the IFN-inhibition by stiffness as noted in Example 5.

FIG. 5B shows that skin stiffness induces NRF2 expression in SSc patients, and CXCL4 overcomes the IFN-inhibition by stiffness as noted in Example 5.

FIG. 5C shows that skin stiffness induces NRF2 expression in SSc patients, and CXCL4 overcomes the IFN-inhibition by stiffness as noted in Example 5.

FIG. 6A shows that inflamed skin stiffness induces NRF2 and inhibits IFN responses in taped stripping mice model as noted in Example 6.

FIG. 6B shows that inflamed skin stiffness induces NRF2 and inhibits IFN responses in taped stripping mice model as noted in Example 6.

FIG. 7A shows that increasing stiffness inhibits IFN-I response in pDCs via integrin-β1. Purified pDCs from Healthy Donors (HDs) were cultured either at 0.2 kPa or 50 kPa for 5 h and analyzed by RNA-sequencing. Principal component analysis (PCA) plot of RNA-seq analysis.

FIG. 7B shows that increasing stiffness inhibits IFN-I response in pDCs via integrin-β1. Purified pDCs from Healthy Donors (HDs) were cultured either at 0.2 kPa or 50 kPa for 5 h and analyzed by RNA-sequencing. Canonical pathway bar chart as identified by QIAGEN Ingenuity Pathway Analysis (IPA), showing the top 11 pathways that were differentially regulated by stiffness in human pDCs.

FIG. 7C shows that increasing stiffness inhibits IFN-I response in pDCs via integrin-β1. Purified pDCs from Healthy Donors (HDs) were cultured either at 0.2 kPa or 50 kPa for 5 h and analyzed by RNA-sequencing. Volcano plot comparing gene expression in pDCs at 0.2 kPa or 50 kPa. Colors on graph indicate differentially expressed genes and the genes involved in cell migration are indicated.

FIG. 7D shows that increasing stiffness inhibits IFN-I response in pDCs via integrin-β1. Purified pDCs from HDs were cultured with media alone or at different stiffness (as indicated) alone or either with the TLR9 agonist (C274 at 0.075 μM) or the TLR7 agonist (Influenza virus, VR-95 at 0.5 pfu/cell). Supernatants were collected after 15 h and IFN-α secretion quantified by ELISA. Individual donors are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 7E shows that increasing stiffness inhibits IFN-I response in pDCs via integrin-β1. Purified pDCs from HDs were cultured with media alone or at different stiffness (as indicated) alone or either with the TLR9 agonist (C274 at 0.075 μM) or the TLR7 agonist (Influenza virus, VR-95 at 0.5 pfu/cell). Supernatants were collected after 15 h and IFN-α secretion quantified by ELISA. Individual donors are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 7F shows that increasing stiffness inhibits IFN-I response in pDCs via integrin-β1. Purified pDCs from HDs were first incubated in media alone or with a neutralizing antibody against integrin-β1 (20 ug/ml) for 1 h, then cultured at 50 kPa stiffness alone or with a TLR9 agonist (C274 at 0.075 μM). Supernatants were collected after 15 h and IFN-α secretion quantified by ELISA. Individual donors are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 7G shows that increasing stiffness inhibits IFN-I response in pDCs via integrin-β1. Purified pDCs from HDs were cultured in media alone or with an integrin-β1 agonist (pyrintegrin: 0.5 or 1 μM) for 1 h, followed by incubation with the TLR9 agonist (C274 at 0.075 μM) for 5 h. RNA were collected and analyzed for IFNA2 expression. Individual donors are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 8A shows that skin-stiffness inhibits IFN-I induction via NRF2 activation. Purified pDCs from HDs were cultured at different stiffness (low stiffness: 0.2 kPa and high stiffness: 50 kPa) in the presence of a TLR9 agonist (C274 at 0.075 μM) for 5 h and analyzed by RNA-sequencing. PCA plot of RNA-seq analysis.

FIG. 8B shows that skin-stiffness inhibits IFN-I induction via NRF2 activation. Purified pDCs from HDs were cultured at different stiffness (low stiffness: 0.2 kPa and high stiffness: 50 kPa) in the presence of a TLR9 agonist (C274 at 0.075 μM) for 5 h and analyzed by RNA-sequencing. Volcano plot showing type I IFN genes.

FIG. 8C shows that skin-stiffness inhibits IFN-I induction via NRF2 activation. Purified pDCs from HDs were cultured at different stiffness (low stiffness: 0.2 kPa and high stiffness: 50 kPa) in the presence of a TLR9 agonist (C274 at 0.075 μM) for 5 h and analyzed by RNA-sequencing. All differential regulated genes were analyzed for transcriptional factors that are either activated or inhibited by stiffness in TLR9-activated pDCs and were identified using QIAGEN IPA.

FIG. 8D shows that skin-stiffness inhibits IFN-I induction via NRF2 activation. Purified pDCs from HDs were cultured at different stiffness (low stiffness: 0.2 kPa and high stiffness: 50 kPa) in the presence of a TLR9 agonist (C274 at 0.075 μM) for 5 h and analyzed by RNA-sequencing. Heatmaps of the genes that are transcriptionally regulated by the transcriptional factor NFE2L2 (NRF2) in FIG. 8C.

FIG. 8E shows that skin-stiffness inhibits IFN-I induction via NRF2 activation. Purified pDCs from HDs were cultured either in media alone or with the NRF2 activator (KI696-5 μM) for 1 h, followed by incubation with the TLR9 agonist (C274-0.075 μM) for 5 h. HMOX1 (marker gene induced by NRF2 activation) gene expression was analyzed by Q-PCR. Individual donors are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 8F shows that skin-stiffness inhibits IFN-I induction via NRF2 activation. pDCs were cultured at a stiffness of 0 kPa or 50 kPa alone or with the NRF2 activator (KI696-5 μM) for 5 h. NRF2 protein levels were assessed by flow cytometry.

FIG. 8G shows that skin-stiffness inhibits IFN-I induction via NRF2 activation. pDCs were cultured at a stiffness of 0 kPa or 50 kPa alone or with the NRF2 activator (KI696-5 μM) for 5 h. NRF2 protein levels were assessed by flow cytometry. Individual donors are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 8H shows that skin-stiffness inhibits IFN-I induction via NRF2 activation. pDCs were cultured at a stiffness of 0 kPa or 50 kPa alone or with the NRF2 activator (KI696-5 μM) for 5 h. NRF2 protein levels were assessed by western blot at a similar time point. Changes in the ratio between NRF2 and R-actin levels as compared to medium alone (normalized as 1) are indicated.

FIG. 8I shows that skin-stiffness inhibits IFN-I induction via NRF2 activation. Purified pDCs from HDs were cultured at a stiffness of 0 kPa or 50 kPa for 1 h, followed by incubation with the TLR9 agonist (C274-0.075 μM) for 5 h. HMOX1 gene expression was analyzed by Q-PCR. Individual donors are indicated; all results are represented as mean SEM; and statistical significance was evaluated using Mann-Whitney U test or one-way ANOVA, Tukey's multiple comparisons test. *, P<0.05; **, P<0.01; ***, P<0.001.

FIG. 8J shows that skin-stiffness inhibits IFN-I induction via NRF2 activation. Purified pDCs from HDs were cultured in media alone or with the NRF2 activator (KI696-5 μM) for 1 h, followed by incubation with the TLR9 agonist (C274-0.07 μM). Supernatant were collected after 15 h and IFNα secretion was analyzed by ELISA. Individual donors are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 8K shows that skin-stiffness inhibits IFN-I induction via NRF2 activation. Purified pDCs from HDs were cultured in media alone or with the NRF2 activator (KI696-5 μM) for 1 h, followed by incubation with the TLR9 agonist (C274-0.075 μM). RNA was collected after 5 h and IFNA2 gene expression was analyzed by Q-PCR. IFNA2 gene expression was analyzed by Q-PCR. Individual donors are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 8L shows that skin-stiffness inhibits IFN-I induction via NRF2 activation. Purified pDCs from HDs were first cultured for 1 h either in media alone or with the NRF2 inhibitor (ML385-2 μM), then cultured at a stiffness of 0 kPa or 50 kPa for another 1 h, followed by incubation with the TLR9 agonist (C274 at 0.075 μM) for 5 h. IFNA2 gene expression was analyzed by Q-PCR. Individual donors are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 9A shows that NRF2 inhibits IFN-I response in pDCs by activating the pentose phosphate pathway. Purified pDCs from HDs were cultured in media alone or with the NRF2 activator (KI696) for 1 h, followed by the TLR9 agonist (C274 at 0.075 μM) for 5 h and analyzed by RNA sequencing. IPA identified pathways that were significantly activated or inhibited by the NRF2 activator (KI696) in TLR9-activated pDCs.

FIG. 9B shows that NRF2 inhibits IFN-I response in pDCs by activating the pentose phosphate pathway. Purified pDCs from HDs were cultured in media alone or with the NRF2 activator (KI696) for 1 h, followed by the TLR9 agonist (C274 at 0.075 μM) for 5 h and analyzed by RNA sequencing. Heatmaps showing the genes that are involved in pentose phosphate pathways.

FIG. 9C shows that NRF2 inhibits IFN-I response in pDCs by activating the pentose phosphate pathway. Graphical representation of the pentose phosphate pathway.

FIG. 9D shows that NRF2 inhibits IFN-I response in pDCs by activating the pentose phosphate pathway. Purified pDCs from HDs were cultured at a stiffness 0 kPa or 50 kPa for 1 h, followed by incubation with the TLR9 agonist (C274 at 0.075 μM) for 5 h. G6PD, and TKT gene expression was analyzed by Q-PCR. Individual donors are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 9E shows that NRF2 inhibits IFN-I response in pDCs by activating the pentose phosphate pathway. Purified pDCs from HDs were cultured in media alone or with the NRF2 activator (KI696) for 1 h, followed by incubation with the TLR9 agonist (C274-0.075 μM) for 5 h. Cells were collected for CUT&RUN assay. Peak changes show the changes in the binding of NRF2 in the promoter of HMOX1, G6PD and TKT genes.

FIG. 9F shows that NRF2 inhibits IFN-I response in pDCs by activating the pentose phosphate pathway. Purified pDCs from HDs were cultured in media alone or with the NRF2 activator (KI696) alone or with sodium pyruvate (10 mM) for 1 h, followed by incubation with the TLR9 agonist for 5 h. IFNA2 gene expression was analyzed by Q-PCR. IFNA2 gene expression was analyzed by Q-PCR. Individual donors are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 9G shows that NRF2 inhibits IFN-I response in pDCs by activating the pentose phosphate pathway. Purified pDCs from HDs were transfected with CRISPR-Cas9 complex targeting for G6PD and TKT on exon 2 and exon 3 respectively using Lipofectamine™ CRISPRMAX reagent and cultured with IL3 (20 ng/ml) for 72 h. Cultured pDCs were treated alone or with the NRF2 activator (KI696) for 1 h, followed by incubation with the TLR9 agonist (C274-0.3 μM) for 5 h. IFNA2 gene expression was analyzed by Q-PCR. Individual donors are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 10A shows that skin stiffness activates NRF2 to inhibit IFN responses and promotes inflammation resolution following mild skin injury using the tape-stripping model. 8-10 weeks old C57BL/6 mice were either shaved only or tape stripped (TAPE). Blood and skin biopsies were collected on day 1 and day 5 after tape stripping. CD45+ cell infiltration in inflamed skin was analyzed by flow cytometry. Individual mice from three different experiments are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 10B shows that skin stiffness activates NRF2 to inhibit IFN responses and promotes inflammation resolution following mild skin injury using the tape-stripping model. 8-10 weeks old C57BL/6 mice were either shaved only or tape stripped (TAPE). Blood and skin biopsies were collected on day 1 and day 5 after tape stripping. Interferon stimulated genes (ISGs) such as IRF7, MX1, ISG20, and ISG15 gene expressions were analyzed by Q-PCR. Individual mice from three different experiments are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 10C shows that skin stiffness activates NRF2 to inhibit IFN responses and promotes inflammation resolution following mild skin injury using the tape-stripping model. 8-10 weeks old C57BL/6 mice were either shaved only or tape stripped (TAPE). Blood and skin biopsies were collected on day 1 and day 5 after tape stripping. pDCs infiltration in inflamed skin was analyzed by flow cytometry. Individual mice from three different experiments are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 10D shows that skin stiffness activates NRF2 to inhibit IFN responses and promotes inflammation resolution following mild skin injury using the tape-stripping model. 8-10 weeks old C57BL/6 mice were either shaved only or tape stripped (TAPE). Blood and skin biopsies were collected on day 1 and day 5 after tape stripping. pDCs (CD45+CD11b−Ly6GCD317+) were sorted from the blood and inflamed skin of TAPE mice. NRF2, MX1 and ISG15 gene expression was analyzed by Q-PCR. (F-M) 8-10 weeks old C57BL/6 mice or NRF2-KO mice were either shaved only or tape stripped (TAPE). Blood and skin biopsies were collected on day 5 after tape stripping. Individual mice from three different experiments are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 10E shows that skin stiffness activates NRF2 to inhibit IFN responses and promotes inflammation resolution following mild skin injury using the tape-stripping model. 8-10 weeks old C57BL/6 mice were either shaved only or tape stripped (TAPE). Blood and skin biopsies were collected on day 1 and day 5 after tape stripping. pDCs (CD45+CD11b−Ly6GCD317+) were sorted from the blood and inflamed skin of TAPE mice. NRF2, MX1 and ISG15 gene expression was analyzed by Q-PCR. (F-M) 8-10 weeks old C57BL/6 mice or NRF2-KO mice were either shaved only or tape stripped (TAPE). Blood and skin biopsies were collected on day 5 after tape stripping. Individual mice from three different experiments are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 10F shows that skin stiffness activates NRF2 to inhibit IFN responses and promotes inflammation resolution following mild skin injury using the tape-stripping model. 8-10 weeks old C57BL/6 mice were either shaved only or tape stripped (TAPE). Blood and skin biopsies were collected on day 1 and day 5 after tape stripping. pDCs infiltration in inflamed skin were analyzed by flow cytometry. Individual mice from three different experiments are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 10G shows that skin stiffness activates NRF2 to inhibit IFN responses and promotes inflammation resolution following mild skin injury using the tape-stripping model. 8-10 weeks old C57BL/6 mice were either shaved only or tape stripped (TAPE). Blood and skin biopsies were collected on day 1 and day 5 after tape stripping. pDCs were sorted from the blood and the inflamed skin of TAPE mice and gene expression level of HMOX1 (NRF2 regulated genes) was quantified by Q-PCR. Individual mice from three different experiments are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 10H shows that skin stiffness activates NRF2 to inhibit IFN responses and promotes inflammation resolution following mild skin injury using the tape-stripping model. 8-10 weeks old C57BL/6 mice were either shaved only or tape stripped (TAPE). Blood and skin biopsies were collected on day 1 and day 5 after tape stripping. pDCs were sorted from the blood and the inflamed skin of TAPE mice and gene expression level of MX1 and ISG15 was quantified by Q-PCR. Individual mice from three different experiments are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 10I shows that skin stiffness activates NRF2 to inhibit IFN responses and promotes inflammation resolution following mild skin injury using the tape-stripping model. 8-10 weeks old C57BL/6 mice were either shaved only or tape stripped (TAPE). Blood and skin biopsies were collected on day 1 and day 5 after tape stripping. pDCs were sorted from the blood and the inflamed skin of TAPE mice and gene expression level of G6PD was quantified by Q-PCR. Individual mice from three different experiments are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 10J shows that skin stiffness activates NRF2 to inhibit IFN responses and promotes inflammation resolution following mild skin injury using the tape-stripping model. 8-10 weeks old C57BL/6 mice were either shaved only or tape stripped (TAPE). Blood and skin biopsies were collected on day 1 and day 5 after tape stripping. Neutrophils infiltration in inflamed skin of TAPE C57BL/6 mice on day-1 and day-5 was analyzed by flow cytometry. Individual mice from three different experiments are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 10K shows that skin stiffness activates NRF2 to inhibit IFN responses and promotes inflammation resolution following mild skin injury using the tape-stripping model. 8-10 weeks old C57BL/6 mice were either shaved only or tape stripped (TAPE). Blood and skin biopsies were collected on day 1 and day 5 after tape stripping. Neutrophils infiltration in inflamed skin of WT or NRF2-KO TAPE mice on day-5 were analyzed by flow cytometry. Individual mice from three different experiments are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 10L shows that skin stiffness activates NRF2 to inhibit IFN responses and promotes inflammation resolution following mild skin injury using the tape-stripping model. 8-10 weeks old C57BL/6 mice were either shaved only or tape stripped (TAPE). Blood and skin biopsies were collected on day 1 and day 5 after tape stripping. Gene expression level of MX1 and ISG15 was analyzed by Q-PCR. Individual mice from three different experiments are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 10M shows that skin stiffness activates NRF2 to inhibit IFN responses and promotes inflammation resolution following mild skin injury using the tape-stripping model. 8-10 weeks old C57BL/6 mice were either shaved only or tape stripped (TAPE). Blood and skin biopsies were collected on day 1 and day 5 after tape stripping. Gene expression level of non-ISGs inflammatory genes such as IL1B was analyzed by Q-PCR. Individual mice from three different experiments are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 11A shows that activation of NRF2 reduces inflammation by dampening IFN responses. 8-10 weeks old C57BL/6 mice were either shaved only or tape stripped (TAPE) and injected i.p with either vehicle or KI696. Blood and skin biopsies were collected on day 1 after tape stripping. CD45+ cell infiltration in inflamed skin were analyzed by flow cytometry. Individual mice from 3 different experiments are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 11B shows that activation of NRF2 reduces inflammation by dampening IFN responses. 8-10 weeks old C57BL/6 mice were either shaved only or tape stripped (TAPE) and injected i.p with either vehicle or KI696. Blood and skin biopsies were collected on day 1 after tape stripping. pDCs infiltration in skin was analyzed by flow cytometry. Individual mice from 3 different experiments are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 11C shows that activation of NRF2 reduces inflammation by dampening IFN responses. 8-10 weeks old C57BL/6 mice were either shaved only or tape stripped (TAPE) and injected i.p with either vehicle or KI696. Blood and skin biopsies were collected on day 1 after tape stripping. pDCs (CD45+CD11b−Ly6G-CD317+) were sorted from the skin of TAPE mice treated with either vehicle or KI696 and gene expression levels of HMOX1 were analyzed by Q-PCR. Individual mice from 3 different experiments are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 11D shows that activation of NRF2 reduces inflammation by dampening IFN responses. 8-10 weeks old C57BL/6 mice were either shaved only or tape stripped (TAPE) and injected i.p with either vehicle or KI696. Blood and skin biopsies were collected on day 1 after tape stripping. pDCs (CD45+CD11b−Ly6G−CD317+) were sorted from the skin of TAPE mice treated with either vehicle or KI696 and gene expression levels of ISGs such as MX1, ISG15, and ADAR were analyzed by Q-PCR. Individual mice from 3 different experiments are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 11E shows that activation of NRF2 reduces inflammation by dampening IFN responses. 8-10 weeks old C57BL/6 mice were either shaved only or tape stripped (TAPE) and injected i.p with either vehicle or KI696. Blood and skin biopsies were collected on day 1 after tape stripping. Gene expression levels of IRF7, MX1, ISG20, ISG15, and OAS2 were analyzed by Q-PCR. Individual mice from 3 different experiments are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 11F shows that activation of NRF2 reduces inflammation by dampening IFN responses. 8-10 weeks old C57BL/6 mice were either shaved only or tape stripped (TAPE) and injected i.p with either vehicle or KI696. Blood and skin biopsies were collected on day 1 after tape stripping. Neutrophils infiltration in inflamed skin of TAPE mice injected with either vehicle or KI696 was analyzed by flow cytometry. Individual mice from 3 different experiments are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 11G shows that activation of NRF2 reduces inflammation by dampening IFN responses. 8-10 weeks old C57BL/6 mice were either shaved only or tape stripped (TAPE) and injected i.p with either vehicle or KI696. Blood and skin biopsies were collected on day 1 after tape stripping. Gene expression levels of IL6, IL1B, and TNFA were analyzed by Q-PCR. Individual mice from 3 different experiments are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 11H shows that activation of NRF2 reduces inflammation by dampening IFN responses. 8-10 weeks old C57BL/6 mice were either shaved only or tape stripped (TAPE) and injected i.p with either vehicle or KI696. Blood and skin biopsies were collected on day 1 after tape stripping. Representative images of H&E-stained skin sections at original magnification 20×. The presence of inflammation spreading in the adipose tissue is indicated by blue arrows and of ulcerations is represented by square bracket for large areas of disepithelization or by black arrow for micro regions of epidermal loss.

FIG. 11I shows that activation of NRF2 reduces inflammation by dampening IFN responses. 8-10 weeks old C57BL/6 mice were either shaved only or tape stripped (TAPE) and injected i.p with either vehicle or KI696. Blood and skin biopsies were collected on day 1 after tape stripping. Average histology parameters scored from 0 to 3 in four skin sections of naive (black), tape+vehicle (green), or tape+KI696 (red) for ulcer score (middle panel), for the degree of adipose tissue inflammation (right panel) and for total score (represented as the average sum of the two histology parameters). Individual mice from 3 different experiments are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 12A shows that CXCL4 counters the NRF2-driven inhibition of pDCs and NRF2 cannot prevent IFN-I in patients with SSc. Single cell RNA-sequencing (sc-RNA-seq) of SSc patients from published paper (Gur et al., 2022) were re-analyzed. Expression of genes of the Hippo pathway in pDCs from the blood or the skin of 56 Healthy donors and 97 SSc patients.

FIG. 12B shows that CXCL4 counters the NRF2-driven inhibition of pDCs and NRF2 cannot prevent IFN-I in patients with SSc. Single cell RNA-sequencing (sc-RNA-seq) of SSc patients from published paper (Gur et al., 2022) were re-analyzed. Differentially regulated transcriptional factors in skin-infiltrating pDCs as analyzed by QIAGEN IPA. Upregulated and downregulated genes are identified in red and blue respectively.

FIG. 12C shows that CXCL4 counters the NRF2-driven inhibition of pDCs and NRF2 cannot prevent IFN-I in patients with SSc. Single cell RNA-sequencing (sc-RNA-seq) of SSc patients from published paper (Gur et al., 2022) were re-analyzed. Expression level of NFE2L2 (low vs high) in pDCs from the blood or the skin of SSc patients.

FIG. 12D shows that CXCL4 counters the NRF2-driven inhibition of pDCs and NRF2 cannot prevent IFN-I in patients with SSc. Single cell RNA-sequencing (sc-RNA-seq) of SSc patients from published paper (Gur et al., 2022) were re-analyzed. Expression of ISGs in pDCs with high or low expression of NFE2L2 from the skin of SSc patients.

FIG. 12E shows that CXCL4 counters the NRF2-driven inhibition of pDCs and NRF2 cannot prevent IFN-I in patients with SSc. Purified pDCs from HDs were cultured at a stiffness of 0 kPa or 50 kPa for 1 h, followed by incubation with the TLR9 agonist (C274 at 0.075 μM) alone or with CXCL4 (0.3 g/ml, 1 g/ml and 3 g/ml) for 15 h. IFNα secretion was analyzed by ELISA. Individual donors are indicated; all results are represented as mean SEM; and statistical significance was evaluated using Mann-Whitney U test. *, P<0.05; **, P<0.01; ***, P<0.001.

FIG. 12F shows that CXCL4 counters the NRF2-driven inhibition of pDCs and NRF2 cannot prevent IFN-I in patients with SSc. Purified pDCs from HDs were cultured at a stiffness of 0 kPa or 50 kPa for 1 h, followed by incubation with the TLR9 agonist (C274 at 0.075 μM) alone or with CXCL4 (0.3 g/ml, 1 g/ml and 3 g/ml) for 15 h. IFN secretion was analyzed by ELISA. Individual donors are indicated; all results are represented as mean±SEM; and statistical significance was evaluated using Mann-Whitney U test. *, P<0.05; **, P<0.01; ***, P<0.001.

FIG. 12G shows that CXCL4 counters the NRF2-driven inhibition of pDCs and NRF2 cannot prevent IFN-I in patients with SSc. 8-10 weeks old C57BL/6 mice were either shaved only or tape stripped (TAPE) and injected i.p with either vehicle or KI696 along with intradermal injection of murine CXCL4 (2 ug per mice). Blood and skin biopsies were collected on day 1 after tape stripping. Gene expression levels of IRF7, MX1, ISG20, and ISG15 were analyzed by Q-PCR. Individual donors are indicated; all results are represented as mean±SEM; and statistical significance was evaluated using Mann-Whitney U test. *, P<0.05; **, P<0.01; ***, P<0.001.

FIG. 13A shows that mechanosensing inhibits pDCs response without impacting cellular viability. Purified pDCs from HDs were cultured in media alone or at increasing stiffness, followed by incubation with the TLR9 agonist (C274 at 0.075 μM). After 5 h of culture, gene expression level of IFNA2 was analyzed by Q-PCR. Individual donors are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 13B shows that mechanosensing inhibits pDCs response without impacting cellular viability. Purified pDCs from HDs were cultured in media alone or at increasing stiffness, followed by incubation with the TLR9 agonist (C274 at 0.075 μM). After 15 h of culture, IL6 secretion was analyzed by ELISA. Individual donors are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 13C shows that mechanosensing inhibits pDCs response without impacting cellular viability. Purified pDCs from HDs were cultured at a stiffness of 0 kPa or 50 kPa, followed by incubation with the TLR9 agonist (C274 at 0.075 μM) for 6 h. Percentage of cell viability was assessed by flow cytometry. Individual donors are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 13D shows that mechanosensing inhibits pDCs response without impacting cellular viability. Purified pDCs from HDs were cultured at a stiffness of 0 kPa or 50 kPa, followed by incubation with the TLR9 agonist (C274 at 0.075 μM) for 6 h. Percentage of apoptotic cells was assessed by flow cytometry. Individual donors are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 13E shows that mechanosensing inhibits pDCs response without impacting cellular viability. Purified pDCs from HDs were cultured at a stiffness of 0 kPa or 50 kPa, followed by incubation with the TLR9 agonist (C274 at either 75 nM or 675 nM) for 15 h. IFNα secretion was analyzed by ELISA. Individual donors are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 13F shows that mechanosensing inhibits pDCs response without impacting cellular viability. RNA-seq of pDCs from HDs (Chaudhary et al. 2022; doi: 10.1084/jem.20221085) was reanalyzed for the expression of integrin genes and represented in Log CPM. Individual donors are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 14A shows that stiffness inhibits pDCs response without the involvement of Hippo Pathway. Our RNA-seq data (Chaudhary et al. 2022; doi: 10.1084/jem.20221085) was re-analyzed for the expression of gene related to Hippo pathway in human pDCs. Individual donors are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 14B shows that stiffness inhibits pDCs response without the involvement of Hippo Pathway. Purified pDCs from HDs were cultured at a stiffness of 0 kPa or 50 kPa, followed by incubation with the TLR9 agonist (C274 at 0.075 μM) alone or with super TDU-131 (100 ng/ml) (YAP inhibitor) for 15 h. IFNα secretion was analyzed by ELISA. Individual donors are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 15A shows that stiffness induces stress-associated transcription factors in human pDCs. Purified pDCs from HDs were cultured at either low stiffness (0.2 kPa) or high stiffness (50 kPa) for 6 h and analyzed by RNA-sequencing. Differentially regulated transcriptional factors were analyzed by QIAGEN Ingenuity Pathway Analysis and upregulated and downregulated genes are indicated in red and blue respectively.

FIG. 15B shows that stiffness induces stress-associated transcription factors in human pDCs. Purified pDCs from HDs were cultured at either low stiffness (0.2 kPa) or high stiffness (50 kPa) for 6 h and analyzed by RNA-sequencing. Heatmaps showing the genes that are transcriptionally regulated by NFE2L2 (NRF2) (Top upregulated transcriptional factor in FIG. 15A).

FIG. 15C shows that stiffness induces stress-associated transcription factors in human pDCs. Purified pDCs from HDs were cultured at a stiffness of 0 kPa, 0.2 kPa, 2 kPa and 50 kPa and gene expression level of XBP1 splicing was analyzed by QPCR. Gene expression level of XBP1 splicing was analyzed by Q-PCR. Individual donors are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 15D shows that stiffness induces stress-associated transcription factors in human pDCs. Purified pDCs from HDs were cultured at a stiffness of 0 kPa, 0.2 kPa, and 50 kPa, followed by incubation with the TLR9 agonist (C274 at 0.075 μM). Gene expression level of XBP1 splicing was analyzed by Q-PCR. Individual donors are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 16A shows that NRF2 activation inhibits IFN-I induction in pDCs via the modulation of the glycolysis pathways. Purified pDCs from HDs were cultured in media alone or with the NRF2 activator (KI696 at 5 μM) for 1 h, followed by incubation with the TLR9 agonist (C274 at 0.075 μM) for 15 h. IL6 secretion was analyzed by ELISA. Individual donors are indicated; all results are represented as mean±SEM; and statistical significance was evaluated using Mann-Whitney U test or one way ANOVA, Tukey's multiple comparisons test. *, P<0.05; **, P<0.01; ***, P<0.001.

FIG. 16B shows that NRF2 activation inhibits IFN-I induction in pDCs via the modulation of the glycolysis pathways. Purified pDCs from HDs were cultured in media alone or with the NRF2 activator (KI696 at 5 μM) for 1 h, followed by incubation with the TLR9 agonist (C274 at 0.075 μM) for 15 h. Cell viability was assessed by flow cytometry after 6 h of culture with the TLR9 agonist alone or with KI696. Individual donors are indicated; all results are represented as mean±SEM; and statistical significance was evaluated using Mann-Whitney U test or one way ANOVA, Tukey's multiple comparisons test. *, P<0.05; **, P<0.01; ***, P<0.001.

FIG. 16C shows that NRF2 activation inhibits IFN-I induction in pDCs via the modulation of the glycolysis pathways. Purified pDCs from HDs were cultured in media alone or with the NRF2 activator (4-octyl itaconate (4-OI)-100 μM) for 1 h, followed by incubation with the TLR9 agonist (C274 at 0.075 μM). Cell viability was assessed by flow cytometry. Individual donors are indicated; all results are represented as mean±SEM; and statistical significance was evaluated using Mann-Whitney U test or one way ANOVA, Tukey's multiple comparisons test. *, P<0.05; **, P<0.01; ***, P<0.001.

FIG. 16D shows that NRF2 activation inhibits IFN-I induction in pDCs via the modulation of the glycolysis pathways. Purified pDCs from HDs were cultured in media alone or with the NRF2 activator (4-octyl itaconate (4-OI)-100 μM) for 1 h, followed by incubation with the TLR9 agonist (C274 at 0.075 μM). After 5 h of culture, gene expression level of HMOX1 was analyzed by Q-PCR. Individual donors are indicated; all results are represented as mean±SEM; and statistical significance was evaluated using Mann-Whitney U test or one way ANOVA, Tukey's multiple comparisons test. *, P<0.05; **, P<0.01; *** P<0.001.

FIG. 16E shows that NRF2 activation inhibits IFN-I induction in pDCs via the modulation of the glycolysis pathways. Purified pDCs from HDs were cultured in media alone or with the NRF2 activator (4-octyl itaconate (4-OI)-100 μM) for 1 h, followed by incubation with the TLR9 agonist (C274 at 0.075 μM). After 5 h of culture, gene expression level of IFNA2 was analyzed by Q-PCR. Individual donors are indicated; all results are represented as mean±SEM; and statistical significance was evaluated using Mann-Whitney U test or one way ANOVA, Tukey's multiple comparisons test. *, P<0.05; **, P<0.01; ***, P<0.001.

FIG. 16F shows that NRF2 activation inhibits IFN-I induction in pDCs via the modulation of the glycolysis pathways. Purified pDCs from HDs were cultured in media alone or with the NRF2 activator (4-octyl itaconate (4-OI)-100 μM) for 1 h, followed by incubation with the TLR9 agonist (C274 at 0.075 μM). After 15 h of culture, IFNα secretion was analyzed by ELISA. Individual donors are indicated; all results are represented as mean±SEM; and statistical significance was evaluated using Mann-Whitney U test or one way ANOVA, Tukey's multiple comparisons test. *, P<0.05; **, P<0.01; ***, P<0.001.

FIG. 16G shows that NRF2 activation inhibits IFN-I induction in pDCs via the modulation of the glycolysis pathways. Purified pDCs from HDs were cultured in media alone or with the NRF2 activator (4-octyl itaconate (4-OI)-100 μM) for 1 h, followed by incubation with the TLR9 agonist (C274 at 0.075 μM). After 15 h of culture, IL6 secretion was analyzed by ELISA. Individual donors are indicated; all results are represented as mean±SEM; and statistical significance was evaluated using Mann-Whitney U test or one way ANOVA, Tukey's multiple comparisons test. *, P<0.05; **, P<0.01; ***, P<0.001.

FIG. 16H shows that NRF2 activation inhibits IFN-I induction in pDCs via the modulation of the glycolysis pathways. Purified pDCs from HDs were cultured either with media alone or with KI696 for 4 h. Intracellular ATP was quantified according to the manufacturer's protocol and normalized to medium. Individual donors are indicated; all results are represented as mean±SEM; and statistical significance was evaluated using Mann-Whitney U test or one way ANOVA, Tukey's multiple comparisons test. *, P<0.05; **, P<0.01; ***, P<0.001.

FIG. 16I shows that NRF2 activation inhibits IFN-I induction in pDCs via the modulation of the glycolysis pathways. Purified pDCs from HDs were cultured in media alone or with the NRF2 activator (KI696) alone or with sodium pyruvate (10 mM) for 1 h, followed by incubation with the TLR9 agonist for 5 h and gene expression level of IL6 was analyzed by Q-PCR. Individual donors are indicated; all results are represented as mean SEM; and statistical significance was evaluated using Mann-Whitney U test or one way ANOVA, Tukey's multiple comparisons test. *, P<0.05; **, P<0.01; ***, P<0.001.

FIG. 16J shows that NRF2 activation inhibits IFN-I induction in pDCs via the modulation of the glycolysis pathways. Purified pDCs from HDs were transfected with CRISPR-Cas9 complex targeting either G6PD or TKT on exon 2 and exon 3 respectively using Lipofectamine™ CRISPRMAX reagent and cultured with IL3 (20 ng/ml) for 72 h. Gene expression level of TKT and G6PD was analyzed by Q-PCR. Individual donors are indicated; all results are represented as mean±SEM; and statistical significance was evaluated using Mann-Whitney U test or one way ANOVA, Tukey's multiple comparisons test. *, P<0.05; **, P<0.01; ***, P<0.001.

FIG. 17A is part of a schematic model showing how the skin environment shapes the biology of pDCs towards either skin inflammation resolution or fibrosis. Injury/inflammatory signals.

FIG. 17B is part of a schematic model showing how the skin environment shapes the biology of pDCs towards either skin inflammation resolution or fibrosis. Following injury/inflammatory signals shown in FIG. 17A, pDCs infiltrate the skin, are activated, and produce IFN-I which activates macrophages.

FIG. 17C is part of a schematic model showing how the skin environment shapes the biology of pDCs towards either skin inflammation resolution or fibrosis. The macrophages activated in FIG. 17C are associated with fibroblast activation and increased collagen production.

FIG. 17D is part of a schematic model showing how the skin environment shapes the biology of pDCs towards either skin inflammation resolution or fibrosis. The fibroblast activation and increased collagen production in FIG. 17D are increases skin stiffness. In normal wound healing, these changes exert negative feedback via engagement of stress pathways such as NRF2 which impacts the metabolism of pDCs to terminate the immune cell response allowing normal wound healing. However, in SSc, the activation status of the cells and the dysregulation of NRF2 and factors such as CXCL4 overcome these regulatory pathways causing persistent fibrosis

### DETAILED DESCRIPTION

The presence of a so-called “IFN-signature,” one of the most robust and prevalent patterns of pathological gene expression in multiple autoimmune diseases, has been observed in the skin and blood of patients with skin lesions or skin fibrosis (Crow et al., 2019; Wenzel and Tuting, 2008). The cellular origin and signaling pathways leading to this chronic IFN-I response are not fully defined, and it is likely that the pathways and cells involved may differ in different disease contexts or model tested, e.g., UV-induced inflammation likely involves cGAS/STING signaling (Skopelja-Gardner et al., 2020), while skin injury triggers the recruitment in the skin and activation of plasmacytoid DCs (pDCs) (Barrat and Su, 2019; Gregorio et al., 2010; Guiducci et al., 2010; Reizis, 2019). Although distinct IFN-inducing pathways may play a role at different stages of the disease, overall, it is clear that difficulties in controlling immune responses to nucleic acids (NA), with a key role for pDCs, likely contribute to the presence of interferon-stimulated genes (ISGs) in the skin or autoimmune patients (Barrat et al., 2016; Barrat and Su, 2019; Furie et al., 2019; Furie et al., 2022; Karnell et al., 2021; Reizis, 2019; Wenzel and Tuting, 2008; Werth et al., 2022). Indeed, the role of pDCs in promoting skin lesions and skin fibrosis has been supported in various mouse models (Ah Kioon et al., 2018; Gerber et al., 2013; Gregorio et al., 2010; Guiducci et al., 2010; Ross et al., 2021) and the depletion/inhibition of pDCs in patients with systemic lupus erythematosus (SLE) and CLE led to reduced expression of ISG in the skin associated with amelioration of skin lesions (Furie et al., 2019; Furie et al., 2022; Karnell et al., 2021; Werth et al., 2022). These data obtained with 2 different drugs targeting pDCs directly links IFN-I produced by pDCs and the occurrence of skin lesions in patients with SLE/CLE. Furthermore, our description that pDCs infiltrate the skin of patients with SSc (Ah Kioon et al., 2018) was recently confirmed in a large single cell study in SSc patients (Gur et al., 2022).

A key challenge has been to understand what controls the chronic activation of pDCs once they infiltrate the skin of patients and how their production of IFN-I can impact the response of other infiltrating immune cells. Cells infiltrating the skin experience significant stiffness, particularly in fibrotic skin where the stiffness can reach up to 50 kPa (Handorf et al., 2015; Lagares et al., 2017), which is about 500 fold higher pressure than that experienced by blood cells (Clark et al., 1996; Harn et al., 2019; Lagares et al., 2017). Furthermore, not only do factors such as stiffness vary between organs, but the forces experienced by cells also vary according to the inflammatory status within tissues (Clark et al., 1996; Harn et al., 2019). Inflammatory mediators such as nucleic acids, cytokines and chemokines, including CXCL4 or CXCL10 (Di Domizio et al., 2020; Du et al., 2022) are also present in the skin and we recently described that these chemokines can modulate the cellular response by pDCs to NA sensing (Du et al., 2022). CXCL4 is present at high concentrations in blood and at sites of tissue injury and inflammation (up to 10 μg/ml in SSc) (Affandi et al., 2022; Ah Kioon et al., 2018; Lande et al., 2019; van Bon et al., 2014; Volkmann et al., 2016). CXCL4 also has a strong pro-fibrotic function in vitro and in vivo (Affandi et al., 2022; Erdem et al., 2007; Nguyen et al., 2019; Silva-Cardoso et al., 2017; Silva-Cardoso et al., 2020a; Silva-Cardoso et al., 2020b; Trocme et al., 2009; Yamamoto et al., 2002) and is produced by pDCs in patients with SSc (Ah Kioon et al., 2018; van Bon et al., 2014).

The effective coordination of various biological and mechanical signals provided by the extracellular matrix (ECM) is essential for cellular functions in tissues (Martino et al., 2018). The mechanical properties of the ECM, such as stiffness, can induce signals, a process known as mechanosensing, that activate cellular pathways and profoundly influence their function (Du et al., 2023; Martino et al., 2018; Wagh et al., 2021). The significance of mechanical stimuli has long been recognized in developmental biology and in organ systems subjected to mechanical stress (e.g., cardiovascular and skeletal systems) (Du et al., 2023). In pathological conditions like atherosclerosis, fibrosis, and the peritumoral environment, alterations in tissue mechanics trigger substantial changes at the cellular level, impacting mechanotransduction components such as adhesion molecules, ion channels, cytoskeletal proteins, and associated signaling pathways (Du et al., 2023; Glitsch, 2019; Martino et al., 2018). How the tissue-scale biophysical cues conferred by skin stiffness on pDCs influence responses remains unclear, while understanding how these forces impact immune cell activation, metabolic reprogramming and downstream effector functions may provide new insights related to regulation of cell function and suggest new approaches for treatment of diseases that involve IFN-I responses in the skin.

Disclosed herein is data demonstrating the surprising role of skin stiffness in suppressing the production of IFN-I by human pDCs. The results disclosed herein show that this effect is mediated by activation of the transcription factor NRF2, leading to the induction of the pentose phosphate pathway and subsequent reduction in pyruvate levels. Using a model of skin injury, the results disclosed herein show that the IFN-I response by the pDCs infiltrating the skin is modulated by NRF2 and that CXCL4, can override the inhibitory effect of stiffness on pDC and allow a shift from skin resolution to chronic inflammation. Overall, this disclosure highlights the mechanism of stiffness-mediated mechanosensing in pDC responses, ultimately influencing the resolution of inflammation.

Hence, the studies disclosed herein identify a novel regulatory mechanism exerted by the skin microenvironment and identify points of dysregulation of this mechanism in patients with skin inflammation.

Data presented in preclinical models but also from human clinical trials have pointed to the role of pDCs as an important player in driving the chronic IFN-I response in the skin (Ah Kioon et al., 2018; Barrat and Su, 2019; Furie et al., 2019; Furie et al., 2022; Gregorio et al., 2010; Guiducci et al., 2010; Karnell et al., 2021; Reizis, 2019; Ross et al., 2021; Wenzel and Tuting, 2008; Werth et al., 2022). As pDCs and the IFN-I they produce in response to nucleic acid sensing by TLRs also participate in wound healing (Gregorio et al., 2010), the challenge is to understand the regulation of pDCs once they reach the skin, a microenvironment with substantially higher stiffness than blood. The studies disclosed herein demonstrate that mechanosensing induced signaling has a dramatic effect on the pDC response to TLR7 or TLR9 signaling by inducing a set of stress-associated transcription factors, particularly NRF2, leading to the inhibition of the IFN-I response. Demonstrated herein is that the engagement of NRF2 triggers the pentose phosphate pathway which dysregulates the metabolic cascade required for the optimal activation of pDCs. Whether the NRF2-IRF7 axis may be at play in other cell types is unclear and should be evaluated in further studies. These data unveil a critical mechanism by which the pDC response is terminated in the skin, allowing for resolution of inflammation and proper wound healing. In patients with SSc, pDCs have an activated status and exposure to the profibrotic cytokine CXCL4, these cells are no longer responsive to mechanosensing, leading to the sustained activation of IFN-I and failure to resolve inflammatory lesions.

Mechanosensing is a well-documented phenomenon in developmental biology and organ systems (Du et al., 2023). It can influence cellular functions, including cell migration and adhesion in mammary epithelial cells (Dupont et al., 2011). The effect of mechanosensing on immune cells is less studied, and nothing is known regarding its impact on pDCs. What the data show is that mechanosensing is an essential regulator of the pDC response and that modulation of the signaling induced by high stiffness can determine whether pDC activation favors normal wound healing or alternatively, progresses toward a sustained IFN-I response which leads to chronic skin lesions. It has previously been observed that the extracellular matrix, highly abundant in fibrotic skin, can initiate mechanosensing through integrin-01 (Emsley et al., 2000). Our data demonstrate that neutralizing integrin-01 rescues IFN-I production, while inducing its signaling inhibited IFN-I response by pDCs, supporting a role for integrin-01 in mechanosensing by pDCs. To define the mechanistic pathways involves in mechanosensing, the classical YAP/TAZ-TEADs pathway (Dupont et al., 2011) was studied herein. Not only are most of the players of this pathway, including YAP1 and TEADs, not expressed in pDCs, including in skin-infiltrating pDCs from SSc patients, but the inhibition of YAP/TEADs binding had no effect, excluding a role of the hippo pathway in mechanosensing in pDCs. This led to exploration of the existence of an alternative pathway induced by mechanosensing in human pDCs and potentially in other immune cells.

Through the transcriptomic analysis disclosed herein, it is identified that stiffness-induced mechanosensing triggers the activation of the transcription factor NRF2, which in turn leads to the suppression of IFN-I production by TLR9-activated pDCs. Although there is evidence that integrin-01 signaling is involved in mechanosignaling in pDCs, further studies will be required to understand how this connects with NRF2 activation. Although some evidence has pointed to NRF2's role in activating the PPP (Baardman et al., 2018; Ding et al., 2021), the link between mechanosensing, NRF2 and the PPP was unexpected. These new findings, combined with our recent description that the engagement of the TCA cycle is critical for optimal pDC function (Chaudhary et al., 2022), points to immunometabolism as a key regulator of pDC function, with defects being associated with disease. Similarly, we previously showed that stress pathways and the UPR regulate pDC responses (Chaudhary et al., 2022). This suggests that stress signals which induce key TFs such XBP-1 or NRF2 are potent regulators of metabolic functions in pDC with defects that are associated with the chronic activation of these cells, something that could be explored in other cell types, such as myeloid cells where the effect of mechanosensing could be different, and other disease contexts.

The findings herein hence provide a mechanistic explanation for the chronic activation of pDCs in certain autoimmune diseases. Because pDCs are so essential for initial responses to pathogens and can quickly infiltrate organs, such as the skin, a key question has been to define how to turn off these cells after the initial burst of IFN-I. The data herein indicate that the sensing of the tissue microenvironment, particularly the cell membrane signals provided by tissue stiffness, is a critical element, and that the normal process of inhibiting pDCs activation following an inflammatory insult is essential for wound healing, but that dysregulation of that response, due to the intrinsic activation status of the cells or the presence in tissue of chemokines such as CXCL4 overcome this resolution process, leading to sustained lesions or fibrosis.

Effective wound healing entails a period of acute inflammation that is then suppressed, with resolution of inflammation allowing for formation of new connective tissue and repair. Mechanisms documented to resolve the inflammatory phase of wound repair include efferocytosis, to clear cell debris, and inhibitory cytokines, such as IL-10 (Soliman and Barreda, 2022). The mechanosensing-induced NRF2-dependent pathway we describe represents an additional and novel mechanism for resolution of cutaneous inflammation. The studies herein point to the chemokine CXCL4, a product of pDCs in SSc, as an important mediator interrupting the NRF2-mediated inhibition of IFN-I production. Interestingly, it was reported that α-SMA+ myofibroblasts have reduced expression of NRF2 in SSc patients (Wei et al., 2017), although the role of NRF2 in fibroblasts might differ to what we observed in pDCs. In addition, NRF2 has been suggested to play a role in controlling fibrosis (Kavian et al., 2018), hence, in addition to therapeutic strategies that directly target pDCs or inhibit the IFN-I pathway, our data suggest that approaches either supporting NRF2 expression or inhibiting CXCL4 may shift pDC function and the skin microenvironment such that they are more favorable to resolution of the pathologic process mediated by pDCs in patients with autoimmune diseases, including SSc and SLE.

Overall, the data disclosed herein report a novel mechanosensing pathway contributing to resolution of inflammatory processes in the skin, identify its key mediators as well as soluble factors that control whether the pDC production of IFN-I is well-regulated or sustained. These observations suggest opportunities to accelerate wound healing as well as strategies to limit the chronic inflammatory and fibrotic processes in patients with autoimmune skin diseases.

In some embodiments, the present disclosure provides, inter alia, a method of inhibiting pDC activity, said method comprising activation of NRF2.

In some embodiments, the pDCs are in skin tissue.

In some embodiments, the pDC is activated by TLR7 and/or TLR9.

In some embodiments, the activation of NRF2 is by exposure to an NRF2 activator.

In some embodiments, the NRF2 activator is KI696.

In some embodiments, the NRF2 activator is 4-octyl itaconate.

In some embodiments, the present disclosure provides a method of inhibiting pDC activity in the skin, said method comprising increasing the stiffness or physical pressure in the skin.

In some embodiments, the method comprises inhibiting the activation of pDCs in the skin.

In some embodiments, the inhibiting step comprises exposing the pDCs in the skin to transcription inhibitors.

In some embodiments, the transcription inhibitor comprises NRF2.

In some embodiments, the pDC is activated by TLR7 and/or TLR9.

In some embodiments, the present disclosure provides, inter alia, a method of inhibiting pDC activity, said method comprising inducing the Pentose Phosphate pathway.

In some embodiments, the Pentose Phosphate pathway is induced by G6PD induction or activation. In some embodiments, the Pentose Phosphate pathway is induced by G6PD induction. In some embodiments, the Pentose Phosphate pathway is induced by G6PD activation.

In some embodiments, the Pentose Phosphate pathway is induced by TKT induction or activation. In some embodiments, the Pentose Phosphate pathway is induced by TKT induction. In some embodiments, the Pentose Phosphate pathway is induced by TKT activation.

In some embodiments, the present disclosure provides, a method of inhibiting pDC activity, said method comprising inducing or activating G6PD. In some embodiments, the present disclosure provides, a method of inhibiting pDC activity, said method comprising inducing G6PD. In some embodiments, the present disclosure provides, a method of inhibiting pDC activity, said method comprising activating G6PD.

In some embodiments, the present disclosure provides, a method of inhibiting pDC activity, said method comprising inducing or activating TKT. In some embodiments, the present disclosure provides, a method of inhibiting pDC activity, said method comprising inducing TKT. In some embodiments, the present disclosure provides, a method of inhibiting pDC activity, said method comprising activating TKT.

In some embodiments, the pDCs are in skin tissue.

In some embodiments, the pDC is activated by TLR7 and/or TLR9.

In some embodiments, the G6PD is induced or activated by exposure to a G6PD inducer. In some embodiments, the G6PD is induced by exposure to a G6PD inducer. In some embodiments, the G6PD is activated by exposure to a G6PD inducer. In some embodiments, the G6PD is activated by exposure to a G6PD activator.

In some embodiments, the TKT is induced or activated by exposure to a TKT inducer. In some embodiments, the TKT is induced by exposure to a TKT inducer. In some embodiments, the TKT is activated by exposure to a TKT inducer. In some embodiments, the TKT is activated by exposure to a TKT activator.

In some embodiments, the method does not comprise blocking the integrin-β1 receptor.

In some embodiments, the method does not comprise blocking actin polymerization.

In some embodiments, the method does not comprise inhibition of NRF2.

In some embodiments, the method does not comprise a supplement of pyruvates.

In some embodiments, the method does not comprise a supplement of CXCL4.

In some embodiments, inhibiting the pDC activity inhibits IFN secretion.

In some embodiments, the present disclosure provides a method of treating a skin disease or disorder associated with inhibition of pDC activity, said method comprising increasing the stiffness or physical pressure of the skin.

In some embodiments, the method comprises inhibiting the activation of pDCs in the skin.

In some embodiments, the inhibiting step comprises exposing the pDCs in the skin to transcription inhibitors.

In some embodiments, the transcription inhibitor comprises NRF2.

In some embodiments, the pDC is activated by TLR7 and/or TLR9.

In some embodiments, the skin disease or disorder is an autoimmune disorder.

In some embodiments, the skin disease or disorder is systemic sclerosis.

In some embodiments, the skin disease or disorder is systemic lupus erythematosus (SLE).

In some embodiments, the present disclosure provides, inter alia, a method of treating a disease or disorder associated with inhibition of pDC activity, said method comprising activation of NRF2.

In some embodiments, the pDC is activated by TLR7 and/or TLR9.

In some embodiments, activating NRF2 induces the Pentose Phosphate pathway.

In some embodiments, activating NRF2 induces or activates G6PD. In some embodiments, activating NRF2 induces G6PD. In some embodiments, activating NRF2 activates G6PD.

In some embodiments, activating NRF2 induces or activates TKT. In some embodiments, activating NRF2 induces TKT. In some embodiments, activating NRF2 activates TKT.

In some embodiments, the activation of NRF2 is by administration of an NRF2 activator.

In some embodiments, the NRF2 activator is KI696.

In some embodiments, the NRF2 activator is 4-octyl itaconate.

In some embodiments, the present disclosure provides, inter alia, a method of treating a disease or disorder associated with inhibition of pDC activity, said method comprising inducing the Pentose Phosphate pathway.

In some embodiments, the present disclosure provides, a method of treating a disease or disorder associated with inhibition of pDC activity, said method comprising inducing or activating G6PD. In some embodiments, the present disclosure provides, a method of treating a disease or disorder associated with inhibition of pDC activity, said method comprising inducing G6PD. In some embodiments, the present disclosure provides, a method of treating a disease or disorder associated with inhibition of pDC activity, said method comprising activating G6PD.

In some embodiments, the present disclosure provides, a method of treating a disease or disorder associated with inhibition of pDC activity, said method comprising inducing or activating TKT. In some embodiments, the present disclosure provides, a method of treating a disease or disorder associated with inhibition of pDC activity, said method comprising inducing TKT. In some embodiments, the present disclosure provides, a method of treating a disease or disorder associated with inhibition of pDC activity, said method comprising activating TKT.

In some embodiments, the pDC is activated by TLR7 and/or TLR9.

In some embodiments, the method does not comprise blocking the integrin-β1 receptor.

In some embodiments, the method does not comprise blocking actin polymerization.

In some embodiments, the method does not comprise inhibition of NRF2.

In some embodiments, the method does not comprise a supplement of pyruvates.

In some embodiments, the method does not comprise a supplement of CXCL4.

In some embodiments, inhibiting the pDC activity inhibits IFN secretion.

In some embodiments, the disease or disorder is an autoimmune disorder or a fibrotic disorder.

In some embodiments, the disease or disorder is an autoimmune disorder.

In some embodiments, the disease or disorder is a fibrotic disorder.

In some embodiments, the disease or disorder is a skin disease or disorder.

In some embodiments, the disease or disorder is fibrosis.

In some embodiments, the disease or disorder is systemic lupus erythematosus (SLE).

In some embodiments, the skin disease or disorder is systemic sclerosis.

In some embodiments, the skin disease or disorder is psoriasis.

In some embodiments, the skin disease or disorder is scleroderma.

In some embodiments, the skin disease or disorder is cutaneous lupus.

In some embodiments, the cutaneous lupus is acute cutaneous lupus (ACLE).

In some embodiments, the cutaneous lupus is chronic cutaneous lupus (discoid lupus).

In some embodiments, the cutaneous lupus is cutaneous lupus erythematosus (CLE).

In some embodiments, the skin disease or disorder is dermatomyositis.

In some embodiments, the skin disease or disorder is Behcet's Disease.

In some embodiments, the skin disease or disorder is ocular cicatricial pemphigoid (OCP).

In some embodiments, the skin disease or disorder is pemphigus.

In some embodiments, the skin disease or disorder is epidermolysis bullosa.

In some embodiments, the skin disease or disorder is epidermolysis bullosa acquisita (EBA).

In some embodiments, the skin disease or disorder is bullous pemphigoid.

In some embodiments, the skin disease or disorder is lichen planus.

In some embodiments, the skin disease or disorder is lichen sclerosus.

It is further appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination. Thus, it is contemplated as features described as embodiments of the compounds of the disclosure can be combined in any suitable combination.

As used herein, the term “administration” typically refers to the administration of a composition to a subject or system. Those of ordinary skill in the art will be aware of a variety of routes that may, in appropriate circumstances, be utilized for administration to a subject, for example a human subject. In some embodiments, administration may involve dosing that is intermittent (e.g., a plurality of doses separated in time) and/or periodic (e.g., individual doses separated by a common period of time) dosing.

The terms “subject,” “individual,” or “patient,” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the methods are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

As disclosed in the Examples, matrigel was used with different stiffness and blocks its effect with inhibitors, activators, CRISPR-Cas9 system, metabolites and cytokines. The following observations were made in the Examples:
1. Increasing stiffness or physical pressure inhibits TLR7/9-activated pDCs, i.e., Example 1.
2. Blocking Integrin-β1 receptor rescues from stiffness-mediated IFN inhibition, i.e., Example 1.
3. Blocking actin polymerization rescues from stiffness-mediated IFN inhibition, i.e., Example 1.
4. Hippo pathway components such as LATS1/2 and TAZ, only express in pDCs but not other components such as YAP1 and TEADs1/2/3/4, i.e., Example 2.
5. Increasing stiffness or physical pressure activates NFE2L2 (NRF2) transcriptional factor, i.e., Example 3.
6. Inhibition of NRF2 rescues from IFN inhibition by stiffness, i.e., Example 3.
7. NRF2 activator inhibits TLR9-activated pDCs, i.e., Example 3.
8. NRF2 activator and stiffness induce Pentose phosphate pathway by inducing glucose 6-phosphate dehydrogenases (G6PD) and inhibiting TLR9-activated pDCs, i.e., Example 4.
9. NRF2 binds to G6PD promoter to induce its expression, i.e., Example 4.
10. Addition of pyruvates rescues from IFN inhibition by NRF2 activator, i.e., Example 4.
11. CXCL4 rescues from IFN inhibition by stiffness or physical pressure, i.e., Example 5.
12. pDCs from SSc patients express low level of NRF2 whereas there is no change in the NRF2 expression in pDCs from SLE patients, i.e., Example 5.
13. pDCs infiltrating into the skin of SSc patients have induced NRF2 expression compared to blood, i.e., Example 6.
14. pDCs infiltrating into the skin of taped stripped have induced NRF2 expression compared to blood, but reduced IFN responses, i.e., Example 6.

Further, the methods disclosed herein allow for the following:
Identification of mechanosensing by skin-stiffness or physical pressure inhibit IFN-I in pDCs.
Identification of the mechanism how mechanosensing regulates IFN-I in pDCs.
Identification of NRF2 inhibitor-ML385 block mechanosensing in pDCs.
Identification of NRF2 activator-KI696 and 4-octyl itaconate as negative regulator of IFN-I in pDCs.
Identification of the mechanism how NRF2 regulates IFN-I in pDCs.
Testing for which pyruvate from TCA cycle regulates NRF2-mediated IFN inhibition in pDCs.

In addition, testing of KI696 or 4-octyl itaconate in a tape stripping mice model can be used to assess each agent's ability to reduce the IFN responses and skin inflammation.

### Combination Therapies

Compounds of the present disclosure, e.g., NRF2 activators such as KI696 or 4-octyl itaconate, or pharmaceutically acceptable salts thereof, can be used in combination with one or more additional therapeutic agents for the treatment of diseases, such as fibrosis, systemic lupus erythematosus (SLE), systemic sclerosis, psoriasis, scleroderma, cutaneous lupus, acute cutaneous lupus (ACLE), chronic cutaneous lupus (discoid lupus), cutaneous lupus erythematosus (CLE), dermatomyositis, Behcet's Disease, ocular cicatricial pemphigoid (OCP), pemphigus, epidermolysis bullosa, epidermolysis bullosa acquisita (EBA), bullous pemphigoid, lichen planus, or lichen sclerosus.

When more than one pharmaceutical agent is administered to a patient, they can be administered simultaneously, separately, sequentially, or in combination (e.g., for more than two agents).

### Formulation, Dosage Forms and Administration

When employed as pharmaceuticals, the compounds of the present disclosure, e.g., NRF2 activators such as KI696 or 4-octyl itaconate, can be administered in the form of pharmaceutical compositions. Thus, the present disclosure provides a composition comprising a compound described herein, or a pharmaceutically acceptable salt thereof, or any of the embodiments thereof, and at least one pharmaceutically acceptable carrier or excipient. These compositions can be prepared in a manner well known in the pharmaceutical art, and can be administered by a variety of routes, depending upon whether local or systemic treatment is indicated and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions for use in the methods herein, which contain, as the active ingredient, a compound of the present disclosure or a pharmaceutically acceptable salt thereof, in combination with one or more pharmaceutically acceptable carriers or excipients. In some embodiments, the composition is suitable for topical administration. In making the compositions of the disclosure, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active compound, soft and hard gelatin capsules, suppositories, sterile injectable solutions and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art see, e.g., WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup and methyl cellulose. The formulations can additionally include: lubricating agents such as talc, magnesium stearate and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one compound described herein, or a pharmaceutically acceptable salt thereof, and at least one pharmaceutically acceptable carrier or excipient. In some embodiments, the composition comprises at least one compound described herein, or a pharmaceutically acceptable salt thereof, and at least one component selected from microcrystalline cellulose, lactose monohydrate, hydroxypropyl methylcellulose and polyethylene oxide. In some embodiments, the composition comprises at least one compound described herein, or a pharmaceutically acceptable salt thereof, and microcrystalline cellulose, lactose monohydrate and hydroxypropyl methylcellulose. In some embodiments, the composition comprises at least one compound described herein, or a pharmaceutically acceptable salt thereof, and microcrystalline cellulose, lactose monohydrate and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo 316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g., Methocel K00LV™). In some embodiments, the polyethylene oxide is polyethylene oxide WSR 1105 (e.g., Polyox WSR 1105™).

In some embodiments, a wet granulation process is used to produce the composition. In some embodiments, a dry granulation process is used to produce the composition.

The compositions can be formulated in a unit dosage form, each dosage containing from about 5 to about 1,000 mg (1 g), more usually about 50 mg to about 400 mg, of the active ingredient. In some embodiments, each dosage contains about 50 mg of the active ingredient. In some embodiments, each dosage contains about 100 mg of the active ingredient. In some embodiments, each dosage contains about 200 mg of the active ingredient. In some embodiments, each dosage contains about 300 mg of the active ingredient. In some embodiments, each dosage contains about 400 mg of the active ingredient.

In some embodiments, the compound is administered to the patient at a daily dose in the range of about 50 mg/day to about 400 mg/day. In some embodiments, the compound is administered to the patient at a daily dose in the range of about 50 mg/day to about 300 mg/day, about 50 mg/day to about 300 mg/day, about 50 mg/day to about 200 mg/day, about 50 mg/day to about 100 mg/day, about 50 mg/day to about 75 mg/day, about 50 mg/day to about 60 mg/day, about 300 mg/day to about 400 mg/day, about 200 mg/day to about 400 mg/day, or about 100 mg/day to about 300 mg/day.

In some embodiments, the compound is administered to the patient at a daily dose of about 50 mg/day. In some embodiments, the compound is administered to the patient at a daily dose of about 100 mg/day. In some embodiments, the compound is administered to the patient at a daily dose of about 200 mg/day. In some embodiments, the compound is administered to the patient at a daily dose of about 300 mg/day. In some embodiments, the compound is administered to the patient at a daily dose of about 400 mg/day. In some embodiments, the compound is administered to the patient at a daily dose of about 500 mg/day. In some embodiments, the compound is administered to the patient at a daily dose of about 750 mg/day. In some embodiments, the compound is administered to the patient at a daily dose of about 1000 mg/day. In some embodiments, the compound is administered to the patient at a daily dose of about 10 mg/day. In some embodiments, the compound is administered to the patient at a daily dose of about 1 mg/day.

In some embodiments, the daily dose is in the range of about 1 mg/day to about 1000 mg/day, about 10 mg/day to about 750 mg/day, about 10 mg/day to about 500 mg/day, about 10 mg/day to about 400 mg/day, about 10 mg/day to about 300 mg/day, about 10 mg/day to about 200 mg/day, about 10 mg/day to about 100 mg/day, about 10 mg/day to about 50 mg/day, about 50 mg/day to about 500 mg/day, about 50 mg/day to about 400 mg/day, about 50 mg/day to about 300 mg/day, about 50 mg/day to about 200 mg/day, or about 50 mg/day to about 100 mg/day. In some aspects, the method includes administering to the patient a single dose of the composition. In some aspects, the method includes administering to the patient multiple doses of the composition. In some aspects, the method includes administering to the patient from 1 to 4 doses of the composition per day.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The active compound may be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms and the like.

The therapeutic dosage of a compound of the present disclosure can vary according to, e.g., the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 g/kg to about 1 g/kg of body weight per day. In some embodiments, the dose range is from about 0.01 mg/kg to about 100 mg/kg of body weight per day. In some embodiments, the dose range is from about 0.02 mg/kg to about 20 mg/kg, about 0.05 mg/kg to about 10 mg/kg, 0.1 mg/kg to about 10 mg/kg, 0.2 mg/kg to about 8 mg/kg, 0.5 mg/kg to about 5 mg/kg, 1 mg/kg to about 5 mg/kg, or 2 mg/kg to about 3 mg/kg of body weight per day. In some embodiments, the dose is about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg of body weight per day.

The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, e.g., about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, e.g., liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, e.g., glycerol, hydroxyethyl cellulose, and the like. In some embodiments, topical formulations contain at least about 0.1, at least about 0.25, at least about 0.5, at least about 1, at least about 2 or at least about 5 wt. % of the compound of the disclosure. The topical formulations can be suitably packaged in tubes of, e.g., 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers or stabilizers will result in the formation of pharmaceutical salts.

The therapeutic dosage of a compound of the present disclosure can vary according to, e.g., the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

### REFERENCES

Affandi, A. J., T. Carvalheiro, A. Ottria, J. J. de Haan, M. A. D. Brans, M. M. Brandt, R. G. Tieland, A. P. Lopes, B. M. Fernandez, C. P. J. Bekker, M. van der Linden, M. Zimmermann, B. Giovannone, C. G. K. Wichers, S. Garcia, M. de Kok, G. Stifano, Y. J. Xu, M. A. Kowalska, M. Waasdorp, C. Cheng, S. Gibbs, S. C. A. de Jager, J. A. G. van Roon, T. Radstake, and W. Marut. 2022. CXCL4 drives fibrosis by promoting several key cellular and molecular processes. Cell Rep 38:110189.
Ah Kioon, M. D., M. Pierides, T. Pannellini, G. Lin, C. F. Nathan, and F. J. Barrat. 2021. Noncytotoxic Inhibition of the Immunoproteasome Regulates Human Immune Cells In Vitro and Suppresses Cutaneous Inflammation in the Mouse. J Immunol 206:1631-1641.
Ah Kioon, M. D., C. Tripodo, D. Fernandez, K. A. Kirou, R. F. Spiera, M. K. Crow, J. K. Gordon, and F. J. Barrat. 2018. Plasmacytoid dendritic cells promote systemic sclerosis with a key role for TLR8. Sci Transl Med 10:
Baardman, J., S. G. S. Verberk, K. H. M. Prange, M. van Weeghel, S. van der Velden, D. G. Ryan, R. C. I. Wust, A. E. Neele, D. Speijer, S. W. Denis, M. E. Witte, R. H. Houtkooper, A. O'Neill L, E. V. Knatko, A. T. Dinkova-Kostova, E. Lutgens, M. P. J. de Winther, and J. Van den Bossche. 2018. A Defective Pentose Phosphate Pathway Reduces Inflammatory Macrophage Responses during Hypercholesterolemia. Cell Rep 25:2044-2052 e2045.
Baird, L., and M. Yamamoto. 2020. The Molecular Mechanisms Regulating the KEAP1-NRF2 Pathway. Mol Cell Biol 40:
Barrat, F. J., K. B. Elkon, and K. A. Fitzgerald. 2016. Importance of Nucleic Acid Recognition in Inflammation and Autoimmunity. Annu Rev Med 67:323-336.
Barrat, F. J., T. Meeker, J. Gregorio, J. H. Chan, S. Uematsu, S. Akira, B. Chang, O. Duramad, and R. L. Coffman. 2005. Nucleic acids of mammalian origin can act as endogenous ligands for Toll-like receptors and may promote systemic lupus erythematosus. J Exp Med 202:1131-1139.
Barrat, F. J., and L. Su. 2019. A pathogenic role of plasmacytoid dendritic cells in autoimmunity and chronic viral infection. J Exp Med 216:1974-1985.
Chaudhary, V., M. D. Ah Kioon, S. M. Hwang, B. Mishra, K. Lakin, K. A. Kirou, J. Zhang-Sun, R. L. Wiseman, R. F. Spiera, M. K. Crow, J. K. Gordon, J. R. Cubillos-Ruiz, and F. J. Barrat. 2022. Chronic activation of pDCs in autoimmunity is linked to dysregulated ER stress and metabolic responses. J Exp Med 219:
Chen, S., Y. Zhou, Y. Chen, and J. Gu. 2018. fastp: an ultra-fast all-in-one FASTQ preprocessor. Bioinformatics 34:i884-i890.
Clark, J. A., J. C. Cheng, and K. S. Leung. 1996. Mechanical properties of normal skin and hypertrophic scars. Burns 22:443-446.
Crow, M. K., M. Olferiev, and K. A. Kirou. 2019. Type I Interferons in Autoimmune Disease. Annu Rev Pathol 14:369-393.
Davies, T. G., W. E. Wixted, J. E. Coyle, C. Griffiths-Jones, K. Hearn, R. McMenamin, D. Norton, S. J. Rich, C. Richardson, G. Saxty, H. M. Willems, A. J. Woolford, J. E. Cottom, J. P. Kou, J. G. Yonchuk, H. G. Feldser, Y. Sanchez, J. P. Foley, B. J. Bolognese, G. Logan, P. L. Podolin, H. Yan, J. F. Callahan, T. D. Heightman, and J. K. Kerns. 2016. Monoacidic Inhibitors of the Kelch-like ECH-Associated Protein 1: Nuclear Factor Erythroid 2-Related Factor 2 (KEAP1:NRF2) Protein-Protein Interaction with High Cell Potency Identified by Fragment-Based Discovery. J Med Chem 59:3991-4006.
Di Domizio, J., C. Belkhodja, P. Chenuet, A. Fries, T. Murray, P. M. Mondejar, O. Demaria, C. Conrad, B. Homey, S. Werner, D. E. Speiser, B. Ryffel, and M. Gilliet. 2020. The commensal skin microbiota triggers type I IFN-dependent innate repair responses in injured skin. Nat Immunol 21:1034-1045.
Ding, H., Z. Chen, K. Wu, S. M. Huang, W. L. Wu, S. E. LeBoeuf, R. G. Pillai, J. D. Rabinowitz, and T. Papagiannakopoulos. 2021. Activation of the NRF2 antioxidant program sensitizes tumors to G6PD inhibition. Sci Adv 7:eabk1023.
Du, H., J. M. Bartleson, S. Butenko, V. Alonso, W. F. Liu, D. A. Winer, and M. J. Butte. 2023. Tuning immunity through tissue mechanotransduction. Nat Rev Immunol 23:174-188.
Du, Y., M. D. Ah Kioon, P. Laurent, V. Chaudhary, M. Pierides, C. Yang, D. Oliver, L. B. Ivashkiv, and F. J. Barrat. 2022. Chemokines form nanoparticles with DNA and can superinduce TLR-driven immune inflammation. J Exp Med 219:
Dupont, S., L. Morsut, M. Aragona, E. Enzo, S. Giulitti, M. Cordenonsi, F. Zanconato, J. Le Digabel, M. Forcato, S. Bicciato, N. Elvassore, and S. Piccolo. 2011. Role of YAP/TAZ in mechanotransduction. Nature 474:179-183.
Emsley, J., C. G. Knight, R. W. Farndale, M. J. Barnes, and R. C. Liddington. 2000. Structural basis of collagen recognition by integrin alpha2beta1. Cell 101:47-56.
Erdem, H., S. Pay, U. Musabak, I. Simsek, A. Dinc, A. Pekel, and A. Sengul. 2007. Synovial angiostatic non-ELR CXC chemokines in inflammatory arthritides: does CXCL4 designate chronicity of synovitis? Rheumatol Int 27:969-973.
Furie, R., V. P. Werth, J. F. Merola, L. Stevenson, T. L. Reynolds, H. Naik, W. Wang, R. Christmann, A. Gardet, A. Pellerin, S. Hamann, P. Auluck, C. Barbey, P. Gulati, D. Rabah, and N. Franchimont. 2019. Monoclonal antibody targeting BDCA2 ameliorates skin lesions in systemic lupus erythematosus. J Clin Invest 129:1359-1371.
Furie, R. A., R. F. van Vollenhoven, K. Kalunian, S. Navarra, J. Romero-Diaz, V. P. Werth, X. Huang, G. Clark, H. Carroll, A. Meyers, C. Musselli, C. Barbey, N. Franchimont, and L. T. Investigators. 2022. Trial of Anti-BDCA2 Antibody Litifilimab for Systemic Lupus Erythematosus. N Engl J Med 387:894-904.
Gerber, E. E., E. M. Gallo, S. C. Fontana, E. C. Davis, F. M. Wigley, D. L. Huso, and H. C. Dietz. 2013. Integrin-modulating therapy prevents fibrosis and autoimmunity in mouse models of scleroderma. Nature 503:126-130.
Ghosh, A. K., and D. E. Vaughan. 2012. PAI-1 in tissue fibrosis. J Cell Physiol 227:493-507.
Glitsch, M. 2019. Mechano- and pH-sensing convergence on Ca(2+)-mobilising proteins—A recipe for cancer? Cell Calcium 80:38-45.
Gomez-Manzo, S., J. Marcial-Quino, A. Vanoye-Carlo, H. Serrano-Posada, D. Ortega-Cuellar, A. Gonzalez-Valdez, R. A. Castillo-Rodriguez, B. Hernandez-Ochoa, E. Sierra-Palacios, E. Rodriguez-Bustamante, and R. Arreguin-Espinosa. 2016. Glucose-6-Phosphate Dehydrogenase: Update and Analysis of New Mutations around the World. Int J Mol Sci 17:
Gregorio, J., S. Meller, C. Conrad, A. Di Nardo, B. Homey, A. Lauerma, N. Arai, R. L. Gallo, J. Digiovanni, and M. Gilliet. 2010. Plasmacytoid dendritic cells sense skin injury and promote wound healing through type I interferons. J Exp Med 207:2921-2930.
Gu, Z., R. Eils, and M. Schlesner. 2016. Complex heatmaps reveal patterns and correlations in multidimensional genomic data. Bioinformatics 32:2847-2849.
Guiducci, C., G. Ott, J. H. Chan, E. Damon, C. Calacsan, T. Matray, K. D. Lee, R. L. Coffman, and F. J. Barrat. 2006. Properties regulating the nature of the plasmacytoid dendritic cell response to Toll-like receptor 9 activation. J Exp Med 203:1999-2008.
Guiducci, C., C. Tripodo, M. Gong, S. Sangaletti, M. P. Colombo, R. L. Coffman, and F. J. Barrat. 2010. Autoimmune skin inflammation is dependent on plasmacytoid dendritic cell activation by nucleic acids via TLR7 and TLR9. J Exp Med 207:2931-2942.
Gur, C., S. Y. Wang, F. Sheban, M. Zada, B. Li, F. Kharouf, H. Peleg, S. Aamar, A. Yalin, D. Kirschenbaum, Y. Braun-Moscovici, D. A. Jaitin, T. Meir-Salame, E. Hagai, B. K. Kragesteen, B. Avni, S. Grisariu, C. Bornstein, S. Shlomi-Loubaton, E. David, R. Shreberk-Hassidim, V. Molho-Pessach, D. Amar, T. Tzur, R. Kuint, M. Gross, O. Barboy, A. Moshe, L. Fellus-Alyagor, D. Hirsch, Y. Addadi, S. Erenfeld, M. Biton, T. Tzemach, A. Elazary, Y. Naparstek, R. Tzemach, A. Weiner, A. Giladi, A. Balbir-Gurman, and I. Amit. 2022. LGR5 expressing skin fibroblasts define a major cellular hub perturbed in scleroderma. Cell 185:1373-1388 e1320.
Handorf, A. M., Y. Zhou, M. A. Halanski, and W. J. Li. 2015. Tissue stiffness dictates development, homeostasis, and disease progression. Organogenesis 11:1-15.
Hao, Y., S. Hao, E. Andersen-Nissen, W. M. Mauck, 3rd, S. Zheng, A. Butler, M. J. Lee, A. J. Wilk, C. Darby, M. Zager, P. Hoffman, M. Stoeckius, E. Papalexi, E. P. Mimitou, J. Jain, A. Srivastava, T. Stuart, L. M. Fleming, B. Yeung, A. J. Rogers, J. M. McElrath, C. A. Blish, R. Gottardo, P. Smibert, and R. Satija. 2021. Integrated analysis of multimodal single-cell data. Cell 184:3573-3587 e3529.
Harn, H. I., R. Ogawa, C. K. Hsu, M. W. Hughes, M. J. Tang, and C. M. Chuong. 2019. The tension biology of wound healing. Exp Dermatol 28:464-471.
https://portals.broadinstitute.org/gpp/public/analysis-tools/%20sgrna-design.
https://sfvideo.blob.core.windows.net/sitefinity/docs/default-source/protocol/alt-r-crispr-cas9-user-guide-ribonucleoprotein-electroporation-amaxa-nucleofector-system6a01611532796e2eaa53ff00001c1b3c.pdf?sfvrsn=71c43407_26.
Jiao, S., C. Li, Q. Hao, H. Miao, L. Zhang, L. Li, and Z. Zhou. 2017. VGLL4 targets a TCF4-TEAD4 complex to coregulate Wnt and Hippo signalling in colorectal cancer. Nature communications 8:14058.
Karnell, J. L., Y. Wu, N. Mittereder, M. A. Smith, M. Gunsior, L. Yan, K. A. Casey, J. Henault, J. M. Riggs, S. M. Nicholson, M. A. Sanjuan, K. A. Vousden, V. P. Werth, J. Drappa, G. G. Illei, W. A. Rees, J. N. Ratchford, and V. I. B. T. Investigators. 2021. Depleting plasmacytoid dendritic cells reduces local type I interferon responses and disease activity in patients with cutaneous lupus. Sci Transl Med 13:
Kavian, N., S. Mehlal, M. Jeljeli, N. E. B. Saidu, C. Nicco, O. Cerles, S. Chouzenoux, A. Cauvet, C. Camus, M. Ait-Djoudi, C. Chereau, S. Kerdine-Romer, Y. Allanore, and F. Batteux. 2018. The Nrf2-Antioxidant Response Element Signaling Pathway Controls Fibrosis and Autoimmunity in Scleroderma. Front Immunol 9:1896.
Korsunsky, I., N. Millard, J. Fan, K. Slowikowski, F. Zhang, K. Wei, Y. Baglaenko, M. Brenner,
P. R. Loh, and S. Raychaudhuri. 2019. Fast, sensitive and accurate integration of single-cell data with Harmony. Nat Methods 16:1289-1296.
Lagares, D., A. Santos, P. E. Grasberger, F. Liu, C. K. Probst, R. A. Rahimi, N. Sakai, T. Kuehl, J. Ryan, P. Bhola, J. Montero, M. Kapoor, M. Baron, X. Varelas, D. J. Tschumperlin, A. Letai, and A. M. Tager. 2017. Targeted apoptosis of myofibroblasts with the BH3 mimetic ABT-263 reverses established fibrosis. Sci Transl Med 9:
Lande, R., E. Y. Lee, R. Palazzo, B. Marinari, I. Pietraforte, G. S. Santos, Y. Mattenberger, F. Spadaro, K. Stefanantoni, N. Iannace, A. M. Dufour, M. Falchi, M. Bianco, E. Botti, L. Bianchi, M. Alvarez, V. Riccieri, M. E. Truchetet, C. L. W. G, C. Chizzolini, and L. Frasca. 2019. CXCL4 assembles DNA into liquid crystalline complexes to amplify TLR9-mediated interferon-alpha production in systemic sclerosis. Nature communications 10:1731.
Langmead, B., and S. L. Salzberg. 2012. Fast gapped-read alignment with Bowtie 2. Nature methods 9:357-359.
Lee, M. J., M. R. Byun, M. Furutani-Seiki, J. H. Hong, and H. S. Jung. 2014. YAP and TAZ regulate skin wound healing. J Invest Dermatol 134:518-525.
Lin, W., Y. Song, T. Li, J. Yan, R. Zhang, L. Han, X. Ba, Y. Huang, K. Qin, Z. Chen, Y. Wang, S. Tu, and Y. Huang. 2023. Triptolide attenuates pulmonary fibrosis by inhibiting fibrotic extracellular matrix remodeling mediated by MMPs/LOX/integrin. Biomed Pharmacother 166:115394.
Ma, Q. 2013. Role of nrf2 in oxidative stress and toxicity. Annu Rev Pharmacol Toxicol 53:401-426.
Mao, X. G., X. Y. Xue, R. Lv, A. Ji, T. Y. Shi, X. Y. Chen, X. F. Jiang, and X. Zhang. 2023. CEBPD is a master transcriptional factor for hypoxia regulated proteins in glioblastoma and augments hypoxia induced invasion through extracellular matrix-integrin mediated EGFR/PI3K pathway. Cell Death Dis 14:269.
Martino, F., A. R. Perestrelo, V. Vinarsky, S. Pagliari, and G. Forte. 2018. Cellular Mechanotransduction: From Tension to Function. Front Physiol 9:824.
Meli, V. S., H. Atcha, P. K. Veerasubramanian, R. R. Nagalla, T. U. Luu, E. Y. Chen, C. F. Guerrero-Juarez, K. Yamaga, W. Pandori, J. Y. Hsieh, T. L. Downing, D. A. Fruman, M. B. Lodoen, M. V. Plikus, W. Wang, and W. F. Liu. 2020. YAP-mediated mechanotransduction tunes the macrophage inflammatory response. Sci Adv 6:
Mills, E. L., D. G. Ryan, H. A. Prag, D. Dikovskaya, D. Menon, Z. Zaslona, M. P. Jedrychowski, A. S. H. Costa, M. Higgins, E. Hams, J. Szpyt, M. C. Runtsch, M. S. King, J. F. McGouran, R. Fischer, B. M. Kessler, A. F. McGettrick, M. M. Hughes, R. G. Carroll, L. M. Booty, E. V. Knatko, P. J. Meakin, M. L. J. Ashford, L. K. Modis, G. Brunori, D. C. Sevin, P. G. Fallon, S. T. Caldwell, E. R. S. Kunji, E. T. Chouchani, C. Frezza, A. T. Dinkova-Kostova, R. C. Hartley, M. P. Murphy, and L. A. O'Neill. 2018. Itaconate is an anti-inflammatory metabolite that activates Nrf2 via alkylation of KEAP1. Nature 556:113-117.
Nguyen, M. V. C., A. Baillet, X. Romand, C. Trocme, A. Courtier, H. Marotte, T. Thomas, M. Soubrier, P. Miossec, J. Tebib, L. Grange, B. Toussaint, T. Lequerre, O. Vittecoq, and P. Gaudin. 2019. Prealbumin, platelet factor 4 and S100A12 combination at baseline predicts good response to TNF alpha inhibitors in rheumatoid arthritis. Joint Bone Spine 86:195-201.
Patra, K. C., and N. Hay. 2014. The pentose phosphate pathway and cancer. Trends Biochem Sci 39:347-354.
Pongor, L. S., J. M. Gross, R. Vera Alvarez, J. Murai, S. M. Jang, H. Zhang, C. Redon, H. Fu, S. Y. Huang, B. Thakur, A. Baris, L. Marino-Ramirez, D. Landsman, M. I. Aladjem, and Y. Pommier. 2020. BAMscale: quantification of next-generation sequencing peaks and generation of scaled coverage tracks. Epigenetics Chromatin 13:21.
Reizis, B. 2019. Plasmacytoid Dendritic Cells: Development, Regulation, and Function. Immunity 50:37-50.
Robinson, J. T., H. Thorvaldsdottir, W. Winckler, M. Guttman, E. S. Lander, G. Getz, and J. P. Mesirov. 2011. Integrative genomics viewer. Nat Biotechnol 29:24-26.
Robinson, M. D., D. J. McCarthy, and G. K. Smyth. 2010. edgeR: a Bioconductor package for differential expression analysis of digital gene expression data. Bioinformatics 26:139-140.
Ross, R. L., C. Corinaldesi, G. Migneco, I. M. Carr, A. Antanaviciute, C. W. Wasson, A. Carriero, J. H. W. Distler, S. Holmes, Y. M. El-Sherbiny, C. S. McKimmie, and F. Del Galdo. 2021. Targeting human plasmacytoid dendritic cells through BDCA2 prevents skin inflammation and fibrosis in a novel xenotransplant mouse model of scleroderma. Ann Rheum Dis 80:920-929.
Silva-Cardoso, S. C., A. J. Affandi, L. Spel, M. Cossu, J. A. G. van Roon, M. Boes, and T. Radstake. 2017. CXCL4 Exposure Potentiates TLR-Driven Polarization of Human Monocyte-Derived Dendritic Cells and Increases Stimulation of T Cells. J Immunol 199:253-262.
Silva-Cardoso, S. C., W. Tao, C. Angiolilli, A. P. Lopes, C. P. J. Bekker, A. Devaprasad, B. Giovannone, J. van Laar, M. Cossu, W. Marut, E. Hack, R. J. de Boer, M. Boes, T. Radstake, and A. Pandit. 2020a. CXCL4 Links Inflammation and Fibrosis by Reprogramming Monocyte-Derived Dendritic Cells in vitro. Front Immunol 11:2149.
Silva-Cardoso, S. C., W. Tao, B. M. Fernandez, M. Boes, T. Radstake, and A. Pandit. 2020b. CXCL4 suppresses tolerogenic immune signature of monocyte-derived dendritic cells. Eur J Immunol 50:1598-1601.
Singh, A., S. Venkannagari, K. H. Oh, Y. Q. Zhang, J. M. Rohde, L. Liu, S. Nimmagadda, K. Sudini, K. R. Brimacombe, S. Gajghate, J. Ma, A. Wang, X. Xu, S. A. Shahane, M. Xia, J. Woo, G. A. Mensah, Z. Wang, M. Ferrer, E. Gabrielson, Z. Li, F. Rastinejad, M. Shen, M. B. Boxer, and S. Biswal. 2016. Small Molecule Inhibitor of NRF2 Selectively Intervenes Therapeutic Resistance in KEAP1-Deficient NSCLC Tumors. ACS Chem Biol 11:3214-3225.
Skopelja-Gardner, S., J. An, J. Tai, L. Tanaka, X. Sun, P. Hermanson, R. Baum, M. Kawasumi, R. Green, M. Gale, Jr., A. Kalus, V. P. Werth, and K. B. Elkon. 2020. The early local and systemic Type I interferon responses to ultraviolet B light exposure are cGAS dependent. Sci Rep 10:7908.
Soliman, A. M., and D. R. Barreda. 2022. Acute Inflammation in Tissue Healing. Int J Mol Sci 24:
Stincone, A., A. Prigione, T. Cramer, M. M. Wamelink, K. Campbell, E. Cheung, V. Olin-Sandoval, N. M. Gruning, A. Kruger, M. Taugeer Alam, M. A. Keller, M. Breitenbach, K. M. Brindle, J. D. Rabinowitz, and M. Ralser. 2015. The return of metabolism: biochemistry and physiology of the pentose phosphate pathway. Biol Rev Camb Philos Soc 90:927-963.
Team, R. C. 2020. R: A language and environment for statistical computing. R Foundation for Statistical Computing, Vienna, Austria. http://www.r-project.org/
Trocme, C., H. Marotte, A. Baillet, B. Pallot-Prades, J. Garin, L. Grange, P. Miossec, J. Tebib, F. Berger, M. J. Nissen, R. Juvin, F. Morel, and P. Gaudin. 2009. Apolipoprotein A-I and platelet factor 4 are biomarkers for infliximab response in rheumatoid arthritis. Ann Rheum Dis 68:1328-1333.
van Bon, L., A. J. Affandi, J. Broen, R. B. Christmann, R. J. Marijnissen, L. Stawski, G. A. Farina, G. Stifano, A. L. Mathes, M. Cossu, M. York, C. Collins, M. Wenink, R. Huijbens, R. Hesselstrand, T. Saxne, M. DiMarzio, D. Wuttge, S. K. Agarwal, J. D. Reveille, S. Assassi, M. Mayes, Y. Deng, J. P. Drenth, J. de Graaf, M. den Heijer, C. G. Kallenberg, M. Bijl, A. Loof, W. B. van den Berg, L. A. Joosten, V. Smith, F. de Keyser, R. Scorza, C. Lunardi, P. L. van Riel, M. Vonk, W. van Heerde, S. Meller, B. Homey, L. Beretta, M. Roest, M. Trojanowska, R. Lafyatis, and T. R. Radstake. 2014. Proteome-wide analysis and CXCL4 as a biomarker in systemic sclerosis. N Engl J Med 370:433-443.
Volkmann, E. R., D. P. Tashkin, M. D. Roth, P. J. Clements, D. Khanna, D. E. Furst, M. Mayes, J. Charles, C. H. Tseng, R. M. Elashoff, and S. Assassi. 2016. Changes in plasma CXCL4 levels are associated with improvements in lung function in patients receiving immunosuppressive therapy for systemic sclerosis-related interstitial lung disease. Arthritis Res Ther 18:305.
Wagh, K., M. Ishikawa, D. A. Garcia, D. A. Stavreva, A. Upadhyaya, and G. L. Hager. 2021. Mechanical Regulation of Transcription: Recent Advances. Trends Cell Biol 31:457-472.
Wei, J., H. Zhu, G. Lord, M. Bhattachayya, B. M. Jones, G. Allaway, S. S. Biswal, B. Korman, R. G. Marangoni, W. G. Tourtellotte, and J. Varga. 2017. Nrf2 exerts cell-autonomous antifibrotic effects: compromised function in systemic sclerosis and therapeutic rescue with a novel heterocyclic chalcone derivative. Translational research: the journal of laboratory and clinical medicine 183:71-86 e71.
Wenzel, J., and T. Tuting. 2008. An IFN-Associated Cytotoxic Cellular Immune Response against Viral, Self-, or Tumor Antigens Is a Common Pathogenetic Feature in “Interface Dermatitis”. J Invest Dermatol 128:2392-2402.
Werth, V. P., R. A. Furie, J. Romero-Diaz, S. Navarra, K. Kalunian, R. F. van Vollenhoven, F. Nyberg, B. H. Kaffenberger, S. Z. Sheikh, G. Radunovic, X. Huang, G. Clark, H. Carroll, H. Naik, F. Gaudreault, A. Meyers, C. Barbey, C. Musselli, N. Franchimont, and L. T. Investigators. 2022. Trial of Anti-BDCA2 Antibody Litifilimab for Cutaneous Lupus Erythematosus. N Engl J Med 387:321-331.
Yamamoto, T., T. Chikugo, and Y. Tanaka. 2002. Elevated plasma levels of beta-thromboglobulin and platelet factor 4 in patients with rheumatic disorders and cutaneous vasculitis. Clin Rheumatol 21:501-504.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### EXAMPLES

### Methods and Materials

Mice: All animal procedures were performed in accordance with the regulations of the Institutional Animal Care and Use Committee of the Hospital for Special Surgery and Weill Cornell Medical College. NRF2 KO-deficient mice.

Purification and culture of pDCs from healthy donors and patients: Enriched leukocytes were obtained from the New York Blood Center (Long Island City, NY) under internal Institutional Review Board-approved protocols and all experiments were approved by the Institutional Biosafety Committee (IBC #: 19-0231) from Weill Cornell Medicine. PBMCs were prepared using Ficoll-Paque density gradient and pDCs were isolated using BDCA4+ positive selection (Miltenyi Biotec: 130-090-532) as previously described (Guiducci et al., 2006). pDCs were cultured at 40,000 cells (for HDs) or at 10,000-20,000 cells (for patients with SSc and SLE) per well in a 96-U bottom plate and incubated at 37° C., 5% CO₂ and 95% humidity. For TLR7 and TLR9 activation assays, pDCs were stimulated with heat-inactivated 2 MOI of H1N1 VR-95 influenza A virus (ATCC) and 0.075 μM of CpG C274 (Guiducci et al., 2006) respectively. In some culture conditions, cells were cultured with Latrunculin A (TOCRIS: 3973), ML385 (Medchemexpress: HY-100525), KI696 (Medchemexpress: HY-101140), 4-octyl itaconate (Medchemexpress: HY-112675), human integrin-β1/CD29 antibody (R&D: MAB17781), sodium pyruvate (Sigma: 8636), human CXCL4 (Sigma-Aldrich: SRP3142), murine CXCL4 (Prepotech: 250-39), pyrintegrin (Medchemexpress: HY-13306) and SuperTDU131 (Medchemexpress: HY-P1728). 96-cell culture plate with various stiffness ranging from 0.2 kPa to 50 kPa were purchased from Matrigen (Catalog No: SW96-HTS-COL-PK).

Flow cytometry and cell sorting: For surface staining or cell sorting, cells were stained with human FcR blocker (Miltenyi Biotec: 130-059-901) or mouse CD16/32 (Biolegend: 101302) with surface marker antibodies: mice CD45 (Biolegend: 103106), mice CD11b (Biolegend: 101216), mice Ly6G (BD Bioscience:560599), mice CD317(eBioscience: 11-3172-82) in 1:100 dilution in FACS buffer for 30 min at 4 C.

For intracellular staining, cultured pDCs were collected, washed in PBS and fixed with Fixation and permeabilization kit (eBioscience: 00-5123-43) for 30 min at room temperature. Fixed pDCs were permeabilized with 0.05% digitonin in FACS buffer for 20 min at room temperature and stained with NRF2 antibody (CST: 14409S; 1:50) for 1 h at room temperature. Cells were washed with FACS buffer and acquired by a fluorescence activated cell sorter (FACS).

For cell viability, cultured pDCs were washed in PBS and resuspended in FACS buffer with DAPI or propidium iodide (Invitrogen: V13243B) to be acquired by a fluorescence activated cell sorter (FACS). For apoptotic cell, pDCs were stained with Annexin V (Biolegend: 640917) in the Annexin staining buffer (Biolegend: 422201) after the cell culture as per the manufacture protocol. Cells were washed with FACS buffer and acquired by a fluorescence activated cell sorter (FACS). The data was analyzed using FlowJo analysis software.

RNA extraction and RT-PCR: pDCs from the cell culture, cell sorting or total skin (Ah Kioon et al., 2018) were lysed for total RNA extraction using the Qiagen RNeasy Plus Mini Kit. Quantity of RNA was measured by Nanodrop, and high-capacity cDNA Reverse Transcription kit (Thermofisher: 4368813) was used to generate cDNA. qPCR reactions were performed. Gene expression levels were calculated based on relative threshold cycle (Ct) values as described (Barrat et al., 2005). This was done using the formula Relative Ct=1000×1.8 (HSK-GENE), where HSK is the mean CT of duplicate housekeeping gene runs (Ubiquitin), GENE is the mean CT of duplicate runs of the gene of interest, and 1000 is arbitrarily chosen as a factor to bring all values above 0. Primers are given in Table 1.

RNA-sequencing and analysis: Total RNA was extracted from cells using the Qiagen RNeasy Plus Mini Kit. All samples were examined for RNA quality by Agilent Bioanalyzer 2100. Illumina libraries were constructed using NEB low input library preparation kit. Multiplexed libraries were generated and pooled at equimolar concentration, and pair-end reads were sequenced on an Illumina NOVASeq 6000 in the Weill Cornell Epigenomics Core Facility at the depth of 21-37 million fragments per sample. Sequencing quality was measured with fastp {Chen, 2018 #30124}. Reads mapped in genes were counted against the human genome (hg38) with STAR aligner and Gencode v21. Differential gene expression analysis was performed in R (Team, 2020 #30127} using the edgeR package {Robinson, 2010 #30125;Gu, 2016 #30126}. Genes with low expression levels (<3 cpm) were filtered from all downstream analyses. The Benjamini-Hochberg false discovery rate procedure was used to calculate the FDR. Genes with p-value<0.05 and log 2 (fold-change)>1 were considered significant. Heatmap were generated by Morpheus packages. Pathways analysis for differential regulated genes were performed in Qiagen Ingenuity Pathway analysis (IPA) and z-scores or p-value were plotted in Prism 9 where z-score 2 represents inhibition and z-score>2 represents activation.

CUT&RUN sequencing and analysis: CUT&RUN-seq library preparation was performed as per the protocol (14-1048). Briefly, 2.5×105 purified pDCs were cultured in 96-well U-bottom plates and left unstimulated or were cultured with KI696 for 1 h, followed by TLR9-ligand treatment for 5 h. Cells were harvested immediately for CUT&RUN-seq library construction. For the CUT&RUN assay, we followed the manufacturer's protocol using CUT&RUN kit (EpiCypher catalog No: 14-1048). To create DNA libraries, we utilized fragmented DNA obtained from the CUT&RUN assay and processed it with the NEBNext Ultra II DNA Library Prep Kit for Illumina (New England Biolabs), adhering to the manufacturer's instructions. Finally, the samples were sequenced using a pair-end 50 bp sequencing in Illumina NOVASeq 6000 platform and analyzed using CUT&RUNTools 2.0 (Fulong Yu, 2022). Briefly, the sequenced reads were aligned to the human genome (hg38) using bowtie2 (Langmead and Salzberg, 2012). And normalized coverage bigwig files relative to the sequencing depth were generated using BAMscale (Pongor et al., 2020). IGV browser from Broad Institute is used to visualize the normalized bigwig files (Robinson et al., 2011).

sc-RNA sequencing analysis: The original scRNASeq data of each sample from GSE195452(Gur et al., 2022) were combined and batch corrected by Harmony (Korsunsky et al., 2019) and analyzed in SeuratV4 (Hao et al., 2021). The cell assignment and patient information were based on the metadata uploaded by the authors (Gur et al., 2022). pDC cluster were the combination of the original pDC and pDC_CXCR4 clusters. ISGs expression were calculated by AddModuleScore function by combining the expression of type I IFN stimulated genes (extracted from the Reactome database interferon alpha/beta signaling pathway). The comparison of pDC numbers in HD and SSc patients. The DEGs between skin and blood of SSc were calculated by FindMakers function with the logfc.threshold=0.25, min.pct=0.25. DEGs between skin and blood of heathy donor shown in the volcano plot were analyzed for transcriptional factor using Qiagen Ingenuity Pathway analysis (IPA) and z-scores were plotted in Prism 9 where z-score<2 represents inhibition and z-score>2 represents activation. Violin plot showing the NFE2L2 expression in Blood and Skin tissues of pDC cells.

Chemokine and Cytokine measurement: Secreted cytokines such as IFN-α (Mabtech: 3425-1H-20) and IL-6 (Mabtech: 3460-1H-20) were quantified in the supernatant of pDC cultures using enzyme-linked immunosorbent assay (ELISA) according to the manufacturer's protocol.

Western Blotting: Cells were lysed in cold lysis buffer (50 mM Tris-HCl pH 7.4, 150 mM NaCl, 1 mM EDTA, 1% (vol/vol) Triton X-100, 2 mM Na3VO4, 1× phosSTOP EASYPACK, 1 mM Pefabloc, and 1×EDTA-free complete protease inhibitor cocktail (Roche, Basel, Switzerland)), and incubated for 30 min on ice. Then, cell debris was pelleted at 13,000 rpm at 4° C. for 10 min. The soluble protein fraction was mixed with 4× Laemmli Sample buffer (BIO-RAD, Cat. #1610747) and 2-mercroptoehanol (BME) (Sigma-Aldrich). Samples for western blotting were subjected to electrophoresis on 4-12% Bis-Tris gels (Invitrogen). Proteins were transferred to polyvinylidene difluoride membrane. Membranes were blocked in 5% (w/v) Bovine Serum Albumin in TBS (20 mm Tris, 50 mm NaCl, pH 8.0) with 0.1% (v/v) Tween-20 (TBST) at room temperature for at least 1 h with shaking at 60 rpm. Membranes were then incubated with primary antibody for NRF2 (CST: 12721) at 4° C. overnight with shaking at 60 rpm. Membranes were washed 3 times in TBST, then probed with anti-rabbit IgG secondary antibodies conjugated to horseradish peroxidase (GE Healthcare, cat: NA9310V and NA9340V) diluted in TBST at room temperature for one hour with shaking at 60 rpm. Next, membranes were washed 3 times in TBST at room temperature with shaking at 60 rpm. Antibody binding was detected using enhanced chemiluminescent substrates for horseradish peroxidase (HRP) (ECL western blotting reagents (PerkinElmer, cat: NEL105001EA), according to the manufacturer's instructions, and visualized using premium autoradiography film (Thomas Scientific, cat: E3018).

ATP detection: ATP determination kit (A22066; Sigma-Aldrich) was used in pDC extracts as per the manufacturer's protocol.

Gene editing in human pDCs: Human pDCs isolated from PBMCs were transfected by adding 150 nM sgRNA-CAS9 ribonucleoprotein complexes to 8×10 cells in suspension using the Lipofectamine™ CRISPRMAX reagent (Thermofisher: 13778075) and cultured in media with IL3 for the pDCs survival. All materials for sgRNA-Cas9 complex generation were purchased from Integrated DNA Technologies and prepared as instructed{https://sfvideo.blob.core.windows.net/sitefinity/docs/default-source/protocol/alt-r-crispr-cas9-user-guide-ribonucleoprotein-electroporation-amaxa-nucleofector-system6a01611532796e2eaa53ff00001c1b3c.pdf?sfvrsn=71c43407_26, #30131}. Eighty hours post-transfection, genetic ablation of target genes was assessed via quantitative RT-PCR. The 20-nucleotide CRISPR-RNA (crRNA) targeting human G6PD (Homo sapiens chromosome 22, GRCh38.p12, NC_001360016) is directed at the genomic sequence 5′-ACGGGCATAGCCCACGATGAAGG-3′ (SEQ ID NO:35) and human TKT (Homo sapiens chromosome 22, GRCh38.p12, NM_001064.4) is directed at the genomic sequence 5′-GACCGGGTGCCCGTCCAAGTCGG-3′ (SEQ ID NO:36) (the 3 additional nucleotides highlighted in bold represent the protospacer adjacent motif, or PAM). This target sequence corresponds to exon 1 of the human G6PD transcript and exon 3 of the human TKT transcript and was manually chosen by identifying a 20-base pair fragment immediately upstream of the highlighted PAM (Lee et al., 2014). The most likely on- and off-target effects of the manually selected CRISPR sequence were then analyzed using the Broad Institute's Genetic Perturbation Platform {https://portals.broadinstitute.org/gpp/public/analysis-tools/%20sgrna-design, #30130}. To validate the genomic editing capacity of the crRNA, quantitative RT-PCR was performed on total RNA isolated from cells transfected with sgRNA-Cas9 complexes containing the G6PD or TKT crRNA as described above. The primers for evaluating deletion efficacy are listed in Table 1.

Tape stripping mice model: Tape stripping was performed after shaving the dorsal area (3×3 cm) and stripping the skin with 10 successive freshly cut 2-3 cm pieces of 3M duct tape on the back of mice, as we have previously described (Yu et al 2022). PBMC were collected from blood, followed by RBC lysis. PBMC from Naïve mice was used as control. Skin biopsy was collected at tape stripping site after 1-days and 5-days for total skin RNA isolation, histology, flow cytometer and cell sorting. Shaved mice were used as control for the total skin RNA isolation. Cell suspensions were prepared by mincing skin, followed by digestion with a collagenase (Sigma-Aldrich: SCR103; concentration:12.5 mg/ml) and liberase TM (Roche: 05401119001; concentration: 100 g/ml) diluted in 1 ml DMEM for 1.5 h at 37° C. The remaining tissue was filtered through a 70-μm strainer (VWR) followed by a 40-μm strainer. Cellular skin infiltrates were characterized by flow cytometry. pDCs were sorted and extracted RNA.

The following markers were used to identify pDCs (CD45hiCD11b−Ly6G−CD317+), and neutrophils (CD45hiCD11b+Ly6G+). For NRF2 activation, 50 μl of Vehicle (5% DMSO, 40% PEG300, 5% Tween-80, 50% PBS) or KI696 (30 mg/kg) was administered by intraperitoneal injection 1 h before tape stripping. Murine CXCL4 (2 μg per mice) were administered by intradermal injection within 1 h of tape stripping.

Pathology: The biopsy specimens were fixed in formalin and embedded in paraffin and sections were stained with hematoxylin-eosin. Blinded evaluation ducted by a pathologist during the course of the study. Multiple organs sections were evaluated for each organ. Inflammation was scored 1 to 4 as follow: 1, minimal; 2, mild; 3, moderate; 4, marked. Sections were counterstained with hematoxylin and evaluated under a DM2000 optical microscope (Leica).

Statistical analysis: All statistical analyses were performed using GraphPad Prism 9 software. Comparisons between two groups were assessed using unpaired or paired (for matched comparisons) two-tailed Student's t-test, or non-parametric Mann-Whitney U-test, or one way ANOVA with Turkey's correction, wherever applicable. Each dot indicates an individual donor or mice. Data are presented as mean±sem. P values of <0.05 were considered to be statistically significant.

TABLE 1

| Genes | Forward Primer Sequences | Reverse Primer Sequence |
|---|---|---|
| Human | CACTTGGTCCTGCGCTTGA (SEQ ID NO: 1) | CAATTGGGAATGCAACAACTTTAT (SEQ ID |
| Ubiquitin | | NO: 2) |
| Human IL6 | TACCCCCAGGAGAAGATTCC (SEQ ID | GCCATCTTTGGAAGGTTCAG (SEQ ID NO: 4) |
| | NO: 3) | |
| Human IFNA2 | CCCAGGAGGAGTTTGGCAA (SEQ ID NO: 5) | TGCTGGATCATCTCATGGAGG (SEQ ID NO: 6) |
| Human NRF2 | TTCCCGGTCACATCGAGAG (SEQ ID NO: 7) | TCCTGTTGCATACCGTCTAAATC (SEQ ID |
| | | NO: 8) |
| Human HMOX1 | AAAGCCCTACAGCAACTGTCG (SEQ ID | AAGACTGCGTTCCTGCTCAAC (SEQ ID NO: 10) |
| | NO: 9) | |
| Human G6PD | AACATCGCCTGCGTTATCCTC (SEQ ID | ACGTCCCGGATGATCCCAA (SEQ ID NO: 12) |
| | NO: 11) | |
| Human TKT | TCATCGAGTGCTACATTGCTG (SEQ ID | GCCATGCGAATCTGGTCAAAG (SEQ ID NO: 14) |
| | NO: 13) | |
| Mouse NRF2 | TAGATGACCATGAGTCGCTTGC (SEQ ID | GCCAAACTTGCTCCATGTCC (SEQ ID NO: 16) |
| | NO: 15) | |
| Mouse HOMX1 | AAGCCGAGAATGCTGAGTTCA (SEQ ID | GCCGTGTAGATATGGTACAAGGA (SEQ ID |
| | NO: 17) | NO: 18) |
| Mouse G6PD | AAATGATGCCTTCCACCAAG (SEQ ID | GCATAGCCCACAATGAAGGT (SEQ ID NO: 20) |
| | NO: 19) | |
| Mouse TKT | ATGGAAGGTTACCATAAGCCAGA (SEQ ID | TGCAGCATGATGTGGGGTG (SEQ ID NO: 22) |
| | NO: 21) | |
| Mouse ISG15 | ACGGTCTTACCCTTTCCAGTC (SEQ ID | CCCCTTTCGTTCCTCACCAG (SEQ ID NO: 24) |
| | NO: 23) | |
| Mouse ISG20 | GTCACGCCTCAGCACATGGT (SEQ ID | CCACCAGCTTGCCTTTCAGAA (SEQ ID NO: 26) |
| | NO: 25) | |
| Mouse MX1 | TCTGTGCAGGCACTATGAGG (SEQ ID | GCCTCTCCACTCCTCTCCTT (SEQ ID NO: 28) |
| | NO: 27) | |
| Mouse IRF7 | ACAGGGCGTTTTATCTTGCG (SEQ ID | TCCAAGCTCCCGGCTAAGT (SEQ ID NO: 30) |
| | NO: 29) | |
| Mouse IL1B | AAACCGTTTTTCCATCTTCTTCTTT (SEQ ID | GACGGCACACCCACCCT (SEQ ID NO: 32) |
| | NO: 31) | |
| Mouse | GCAAGTGGCTAGAGTGCAGAGTAA (SEQ | TGGCTATTAATTATTCGGTCTGCAT (SEQ ID |
| Ubiquitin | ID NO: 33) | NO: 34) |

### Example 1. Increasing Stiffness or Physical Pressure Inhibits Interferon Via Integrin-β1

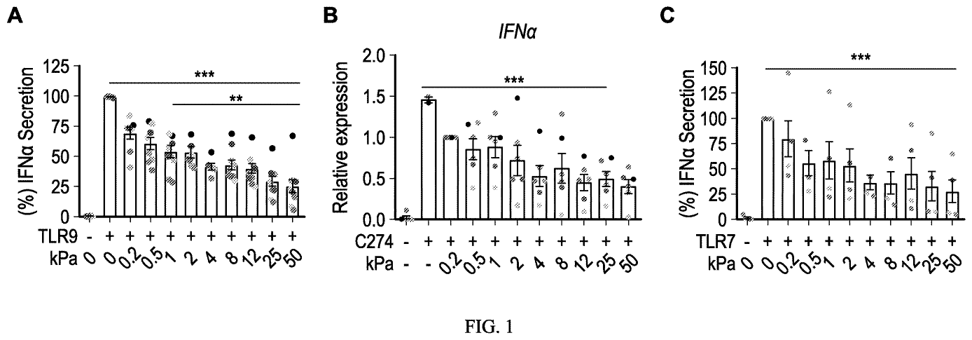

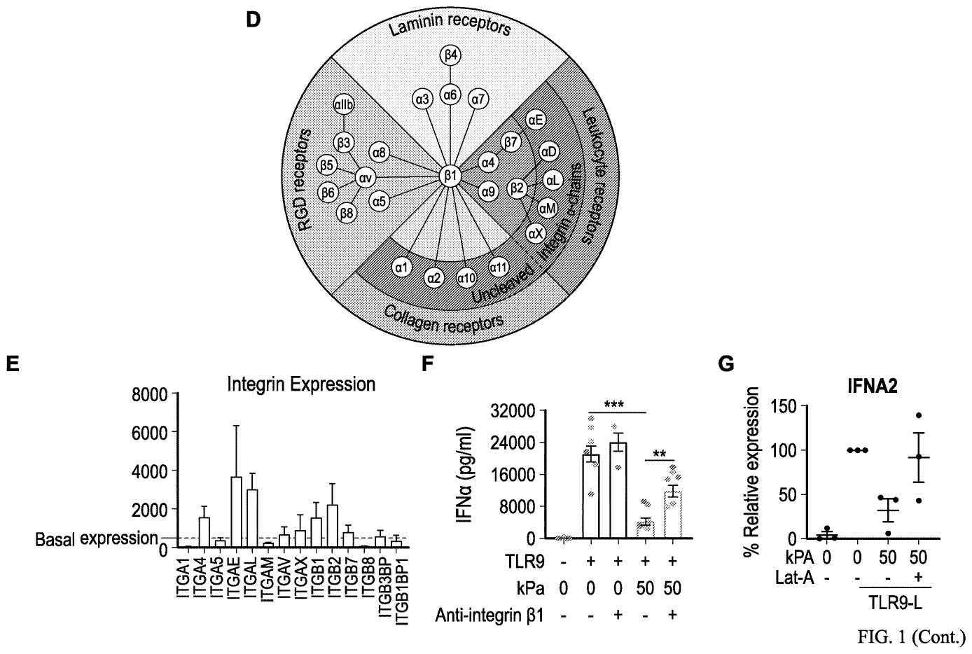

(FIGS. 1A-1C): Purified pDCs from Healthy Donor (HD) were cultured in media alone or with increasing stiffness, followed by TLR9 agonist (C274-0.075 μM) or TLR7 agonist (FLU: 0.5 pfu/ml). (FIGS. 1A and 1C): Supernatant were collected after 15 h of TLR7/9 agonist and analyzed for IFNα secretion via ELISA. (FIG. 1B): RNA were collected after 5 h of TLR9 agonist treatment and analyzed for IFNα expression. (FIG. 1D): Graphical representation of receptors and its subunit. (FIG. 1E): Expression of different integrin subunit in human pDCs. (FIG. 1F): Purified pDCs were cultured in media alone or with neutralizing antibody for Integrin-β1 for 1 h, followed by stiffness for 1 h, and then TLR9 agonist (C274-0.075 μM). Supernatant were collected after 15 h of TLR9 agonist and analyzed for IFNα secretion via ELISA. (FIG. 1G): Purified pDCs were cultured in media alone or with stiffness for 1 h, followed by TLR9 agonist (C274-0.075 μM) or actin polymerization inhibitor (Latrunculin A: 100 nM). RNA were collected after 5 h of TLR9 agonist treatment and analyzed for IFNA2 expression.

### Example 2. Hippo Pathway Components are Absent in Human pDCs

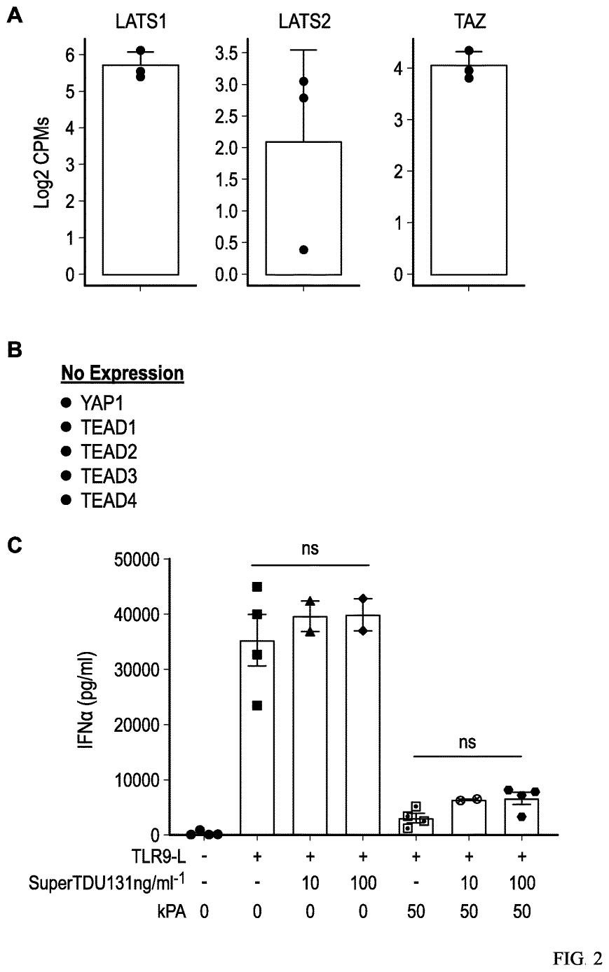

(FIGS. 2A-2B): Purified pDCs from Healthy Donor (HD) were cultured in media alone for 6 h. RNA were collected and preformed RNA-seq. (a) Expression of genes in Log CPM (b) list of genes of Hippo pathway which are not expressed in human pDCs. (FIG. 2C): Purified pDCs from Healthy Donor (HD) were cultured in media alone or with stiffness, followed by TLR9 agonist (C274-0.075 μM) or SuperTDU-131: peptide to block the binding of YAP1 with TEAD3. Supernatant were collected after 15 h of TLR9 agonist and analyzed for IFNα secretion via ELISA.

### Example 3. Stiffness of Skin Inhibits Interferon Via Activation of NRF2 Transcriptional Factor

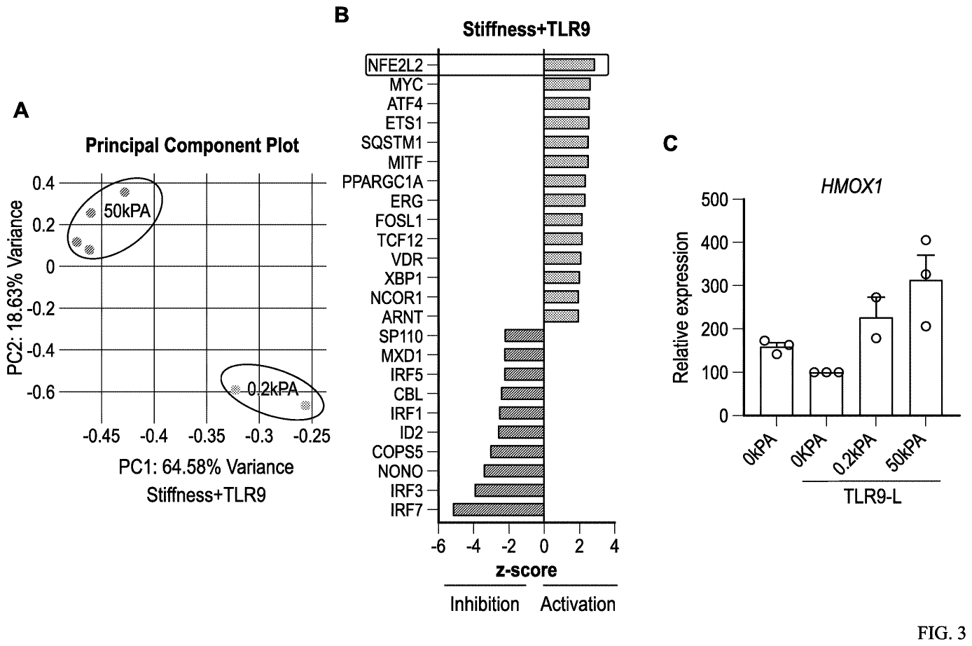

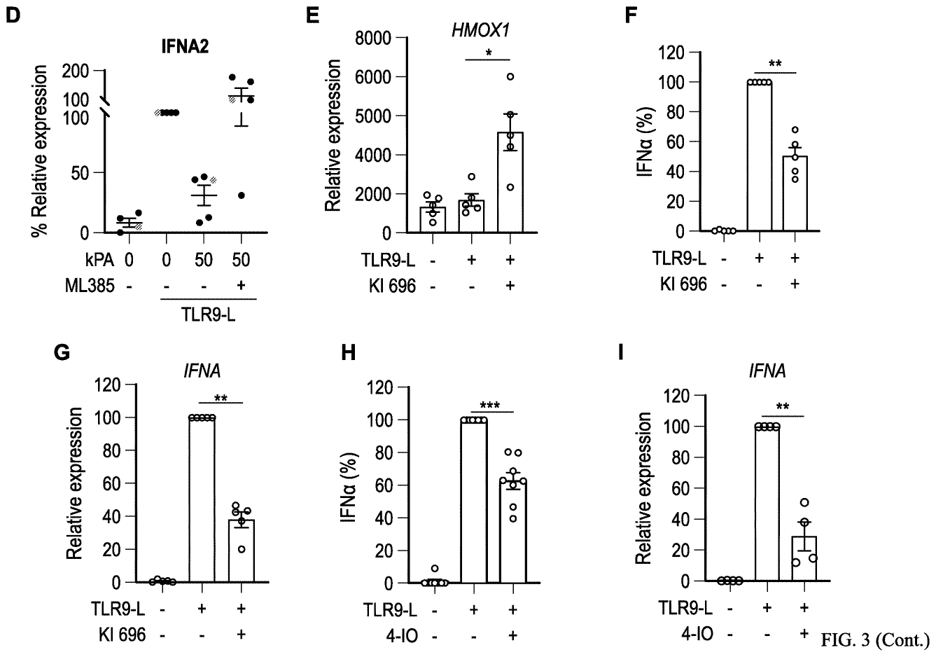

(FIGS. 3A-3B): Purified pDCs from Healthy Donor (HD) were cultured with different stiffness (low stiffness: 0.2 kPA and High stiffness: 50 kPA), followed by TLR9 agonist (C274-0.075 μM)). RNA were collected after 5 h of TLR9 agonist treatment and preformed RNA-seq. (FIG. 3A): PCA plot of RNA-seq analysis. (FIG. 3B): All transcriptional factor that are activated and inhibited by stiffness in TLR9-activated pDCs were identified by transcriptional factors-pathway analysis using QIAGEN Ingenuity Pathway Analysis. (FIG. 3C): Purified pDCs were cultured in media alone or with stiffness, followed by TLR9 agonist (C274-0.075 μM). RNA were collected after 5 h of TLR9 agonist treatment and analyzed for HMOX1 (marker gene induced by NRF2 activation) expression. (FIG. 3D): Purified pDCs were cultured in media alone or with NRF2 inhibitor (ML385-2 μM) for 1 h, followed by stiffness for 1 h, and then TLR9 agonist (C274-0.075 μM). RNA were collected after 5 h of TLR9 agonist treatment and analyzed for IFNA2 expression. (FIGS. 3E-3I): Purified pDCs were cultured in media alone or with NRF2 activator (KI696-5 μM or 4-octyl itaconate (4-01)-100 μM) for 1 h, followed by TLR9 agonist (C274-0.075 μM). (FIG. 3E): RNA were collected after 5 h of TLR9 agonist treatment and analyzed for HMOX1 expression. (FIGS. 3F and 3H): Supernatant were collected after 15 h of TLR9 agonist and analyzed for IFNα secretion via ELISA. (FIGS. 3G and 3I): RNA were collected after 5 h of TLR9 agonist treatment and analyzed for IFNA2 expression.

### Example 4. NRF2 Activates Glucose 6-Phosphate Dehydrogenases by Binding to its Promoter and Inhibits Interferon

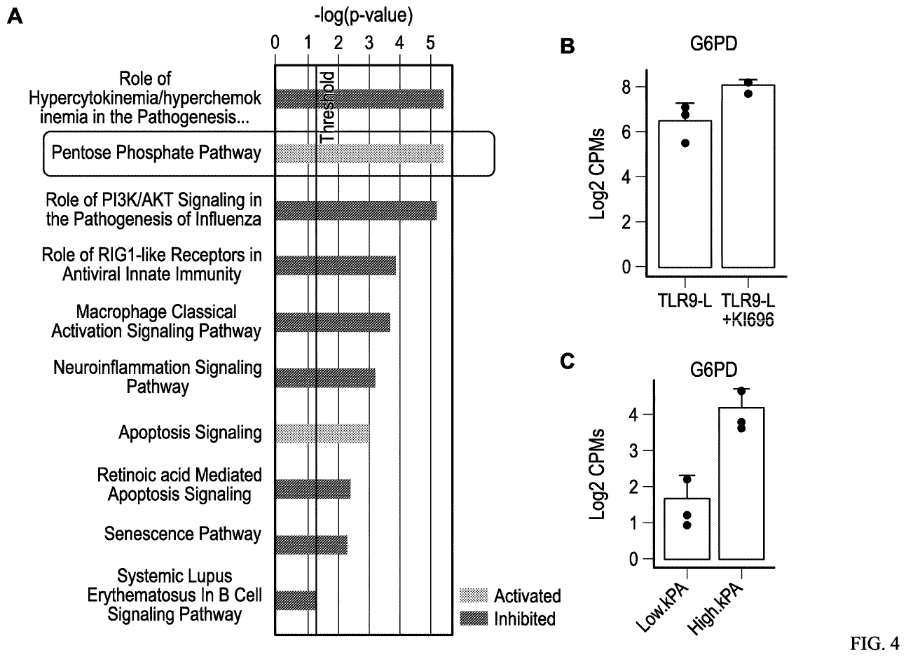

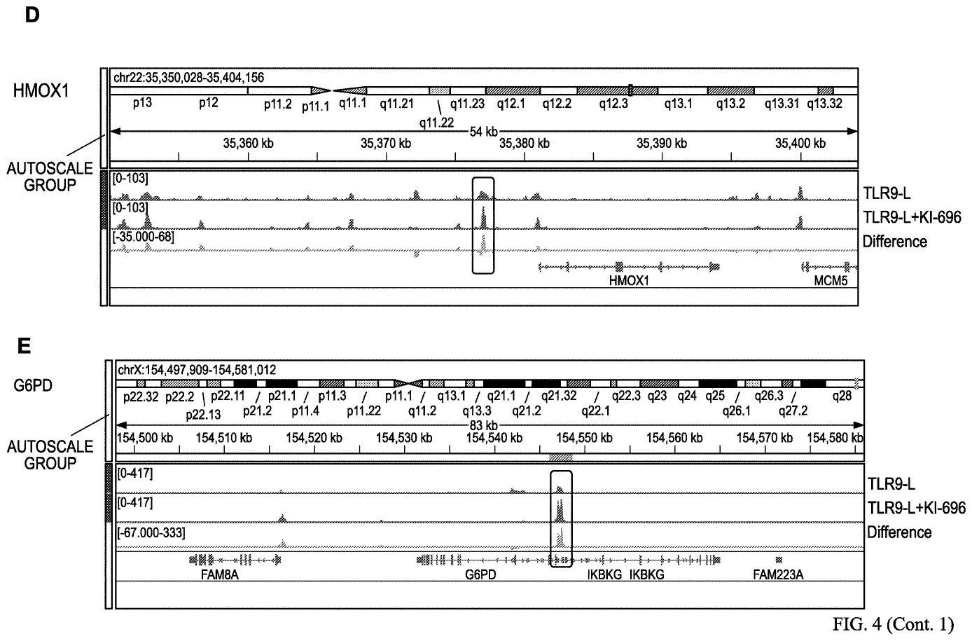

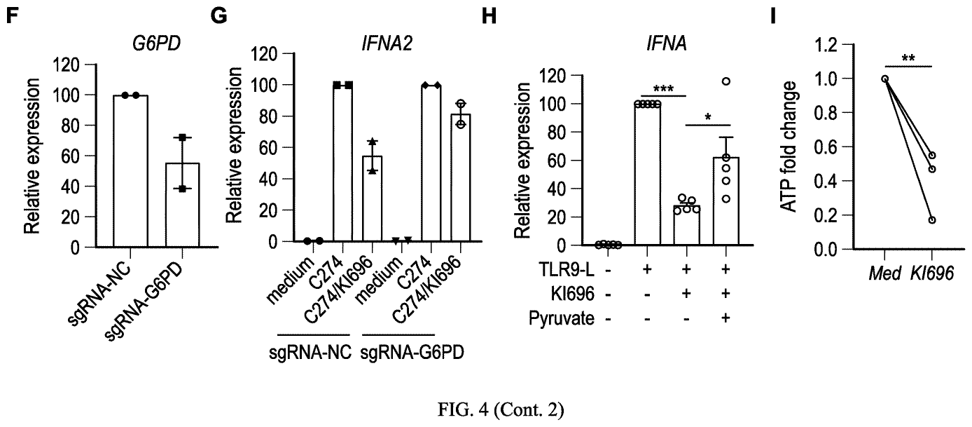

(FIG. 4A) Purified pDCs were cultured in media alone or with NRF2 activator (KI696-5 μM) for 1 h, followed by TLR9 agonist (C274-0.075 μM). (FIG. 4E): RNA were collected after 5 h of TLR9 agonist treatment and preformed RNA-seq. All upregulated and downregulated genes (Fold change:1 and FDR: 0.05) were analyzed for pathway analysis using QIAGEN Ingenuity Pathway Analysis. (FIG. 4B): Gene expression of G6PD in TLR9-activated pDCs with or without NRF2 activator (KI696-5 μM). (FIG. 4C): Gene expression of G6PD in TLR9-activated pDCs in different stiffness (low stiffness: 0.2 kPA and High stiffness: 50 kPA). (FIGS. 4D-4E): Purified pDCs were cultured in media alone or with NRF2 activator (KI696-5 μM) for 1 h, followed by TLR9 agonist (C274-0.075 μM). (FIG. 4F): Purified pDCs were transfected with CRISPR-Cas9 complex targeting for G6PD gene on exon 2 using Lipofectamine™ CRISPRMAX reagent. After transfection, pDCs were cultured with IL3 (20 ng/ml) for 72 h and treated with KI696 for 1 h, followed by TLR9 agonist (C274-0.3 μM) activation for 5 h. RNA were collected and analyzed for G6PD (FIG. 4F) and IFNA2 (FIG. 4G) expression. (FIG. 4H): pDCs were cultured with KI696 with or without sodium pyruvate (10 mM) for 1 h, followed by TLR9 agonist for 5 h and analyzed for IFNA2 expression. (FIG. 4I): pDCs were cultured with or without KI696 for 4 h. Cells were collected and analyzed for Intracellular ATP using ATP determination assay kit (Thermofisher: A22066).

### Example 5. Skin Stiffness Induces NRF2 Expression in SSc Patients, and CXCL4 Overcomes the IFN-Inhibition by Stiffness

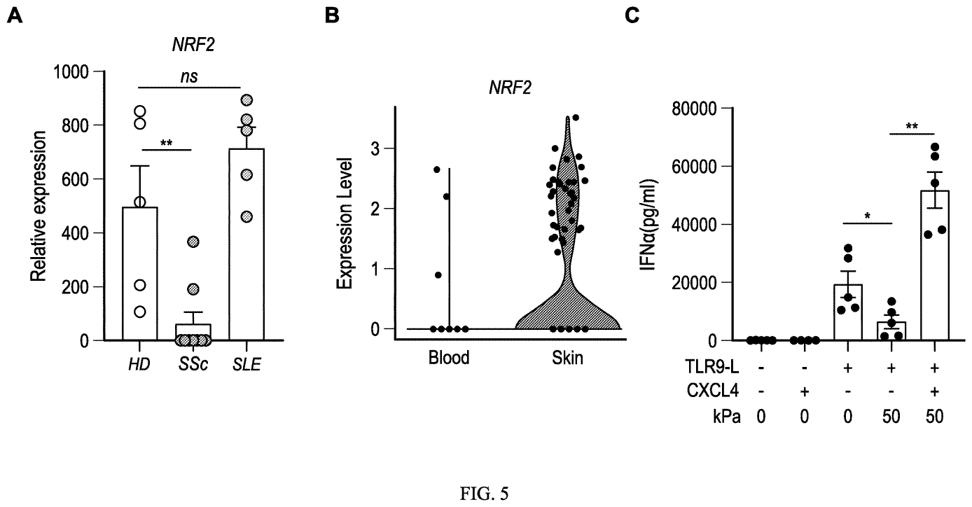

(FIG. 5A): pDCs were purified from fresh blood of healthy donors, SSc and SLE patients. RNA were collected and analyzed for expression of NRF2. (FIG. 5B): Single cell RNA-seq of SSc patients from published paper (Gur et al., 2022, Cell) were re-analysed for NRF2 expression in pDCs population in blood and skin of the 97 SSc patients. (FIG. 5C): Purified pDCs were cultured in media alone or with stiffness (50 kPA) for 1 h, followed by TLR9 agonist (C274-0.075 μM) with/without CXCL4 (3 g/ml) for 15 h. Supernatant were collected after 15 h of TLR9 agonist and analyzed for IFNα secretion via ELISA.

### Example 6. Inflamed Skin Stiffness Induces NRF2 and Inhibit IFN Responses in Tape Stripping Mice Model

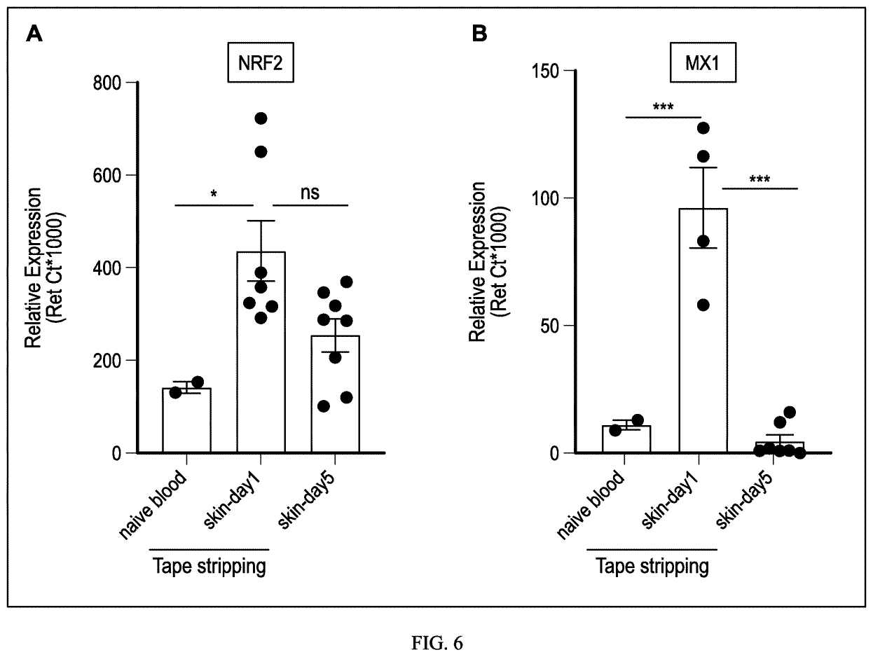

Age-matched C57BL/6 mice were either shaved only or tape stripped (tape stripping). Blood and skin biopsies were collected on day 1 and day 5 after the tape stripping. pDCs (CD45⁺CD11b⁻Ly6G⁻CD317⁺) from blood and inflamed skin of tape stripped mice were sorted. RNA were collected from the sorted pDCs from blood and skin and analyzed for gene expression of NRF2 and Interferon stimulated genes such as MXL. Results are shown in FIGS. 6A and 6B.

Testing of KI696 in a tape stripping mice model to reduce the IFN responses and skin inflammation.

### Example 7. Stiffness-Mediated Mechanosensing Inhibits IFN-I Response by pDCs to TLR7 and TLR9 Signaling

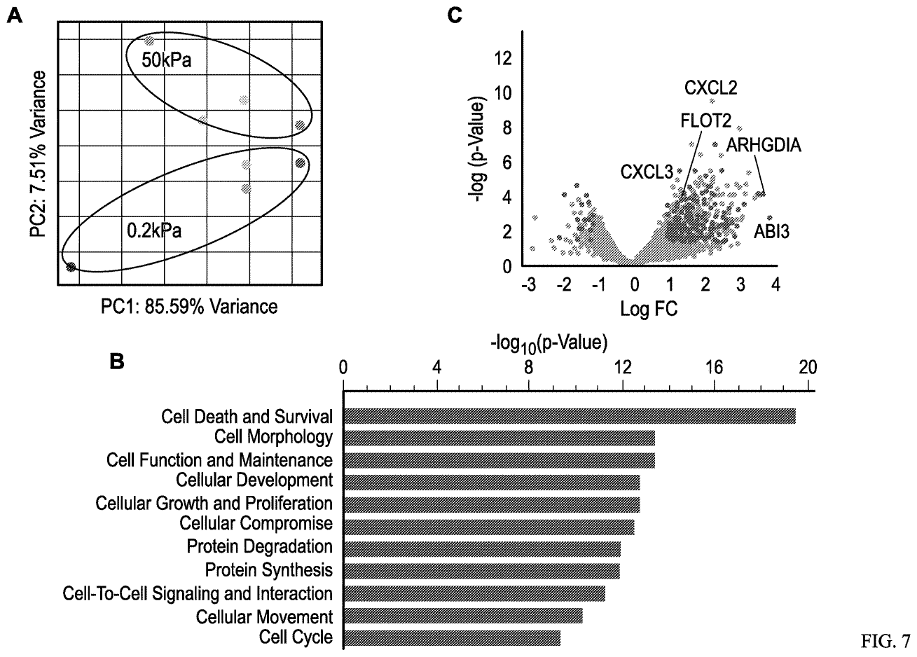

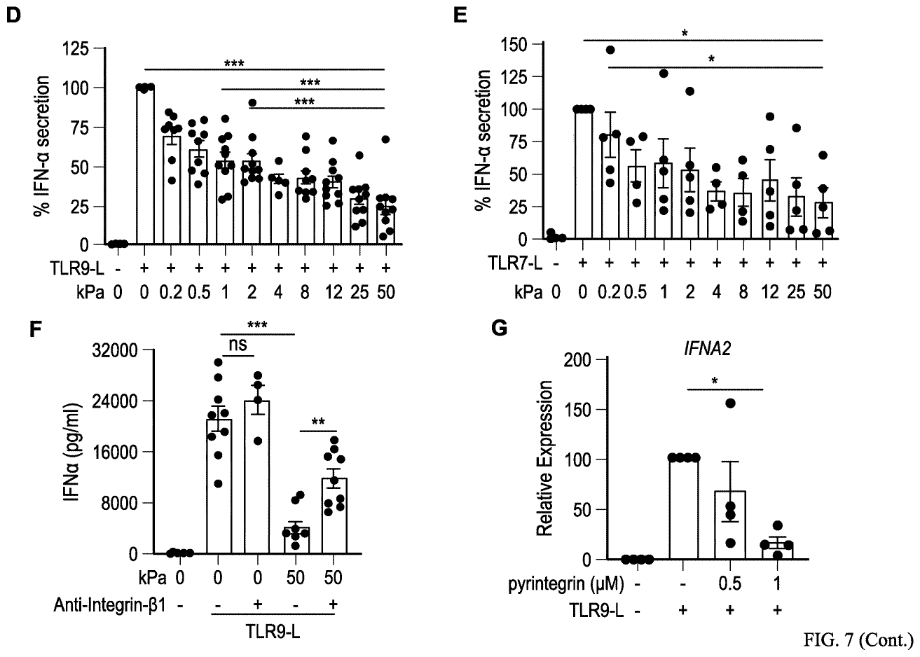

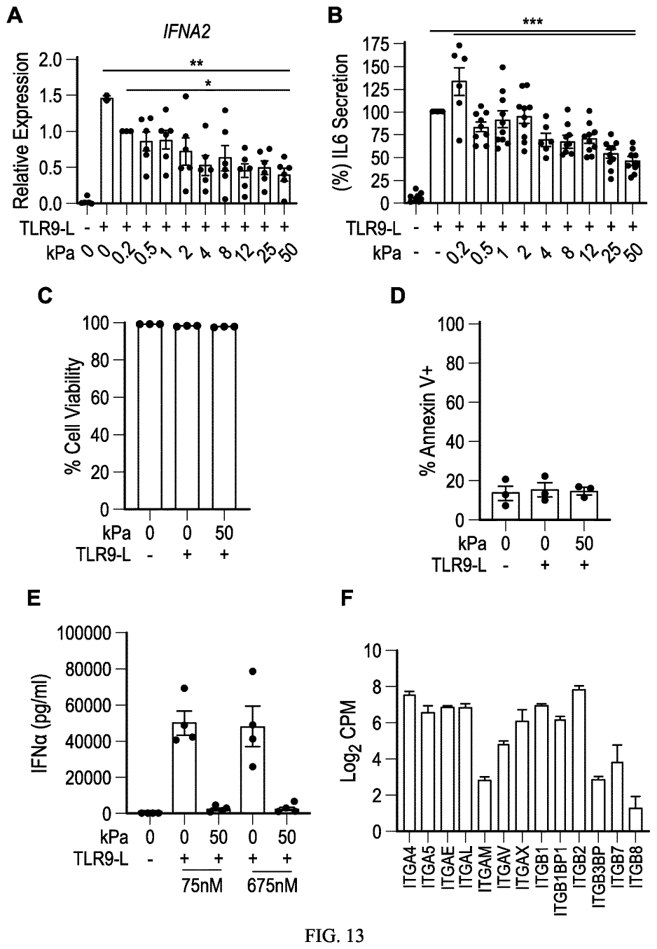

Although the presence of pDCs in the skin of patients with autoimmune diseases is well described and clinically relevant, there is limited understanding of how pDCs respond to nucleic acids under conditions associated with the distinct microenvironment of the skin and under the influence of inflammation. To mimic degrees of stiffness experienced by cells infiltrating healthy or fibrotic skin, hydrogel substrates with a wide range of stiffness (ranging from 0.2 to 50 kPa) that were coated with type I collagen, which is known to be excessively deposited during skin fibrosis (Ghosh and Vaughan, 2012), were employed. Results showed that changes in stiffness, comparing 0.2 kPa and 50 kPa, had a strong impact on the transcriptional landscape of human pDCs (FIG. 7A). A transcriptomic analysis revealed a noteworthy influence of stiffness on pathways associated with cell movement function within pDCs (FIG. 7B). A set of genes associated with cell movement function, such as CXCL2, CXCL3, FLOT2, ARHGDIA, and ABI3, were induced in pDCs under high stiffness (FIG. 7C). As chronic pDC-induced IFN-I has been associated with skin fibrosis, one would hypothesize that increased stiffness would promote sustained IFN-I response by pDCs. Surprisingly, increasing stiffness led to reduced IFN-α production by TLR9- or TLR7-activated pDCs, both at the protein and mRNA levels (FIGS. 7D and 7E and 13A). A similar effect was also observed on IL6 (FIG. 13B) without affecting pDC viability (FIGS. 13C and 13D). To further understand the robustness of this inhibition, CpG-274 at increased concentration (9-fold higher) with similar effect of stiffness (FIG. 13E) was employed. The dimer form of integrin-β1, along with other integrin subunits, interacts with type I collagen (Emsley et al., 2000). Results showed that integrin-01 was expressed on pDCs (FIG. 13F) and that blocking integrin-01 with a neutralizing antibody was able to rescue IFN-α production by pDCs on high stiffness substrates (FIG. 7F), while an agonist for the integrin-01 receptor (pyrintegrin) (Lin et al., 2023; Mao et al., 2023) inhibited IFN-α production by TLR9 activation (FIG. 7G), demonstrating that the sensing of collagen coated on these hydrogels by integrin-β1 plays a role in this effect. Hence, these results highlight the inhibitory impact of mechanical signals on pDC function and raise new questions as to the mechanism associated with this inhibition and how these observations fit with the known chronic activation of pDCs in the skin of patients with autoimmune diseases.

### Example 8: Skin Stiffness Inhibits IFN-I Production Via Transcriptional Activation of NRF2

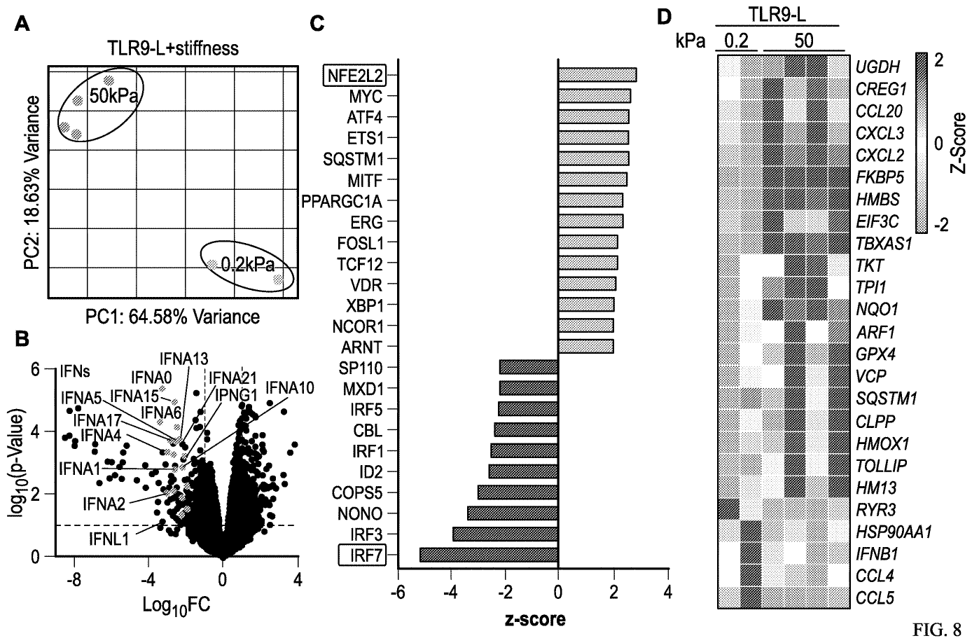

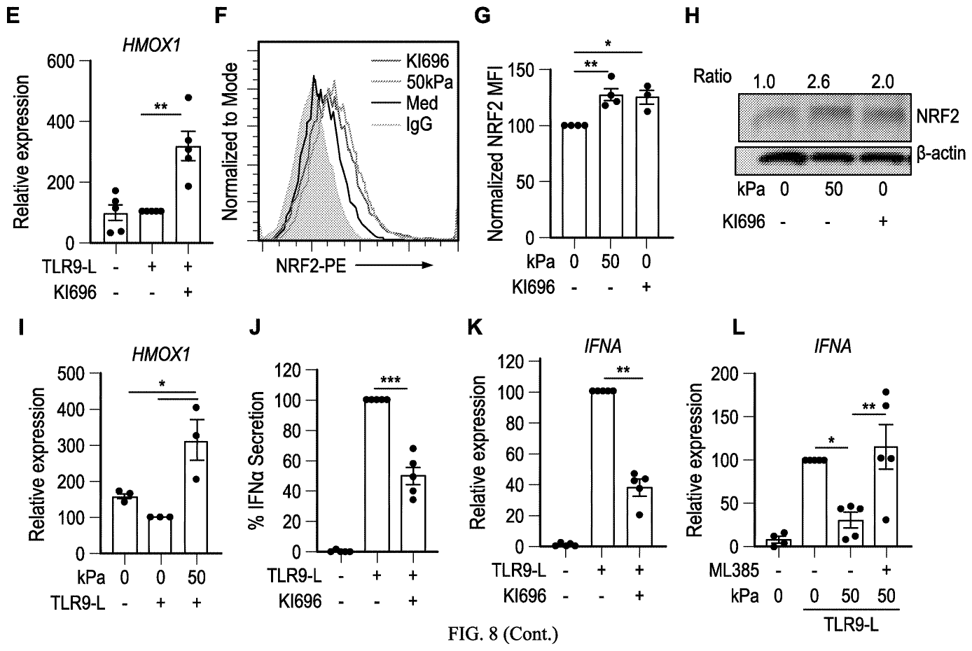

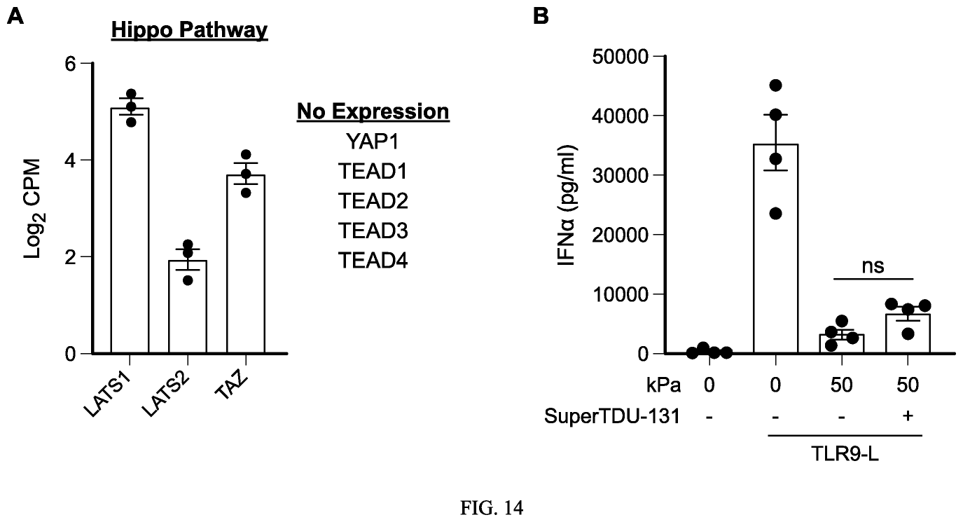

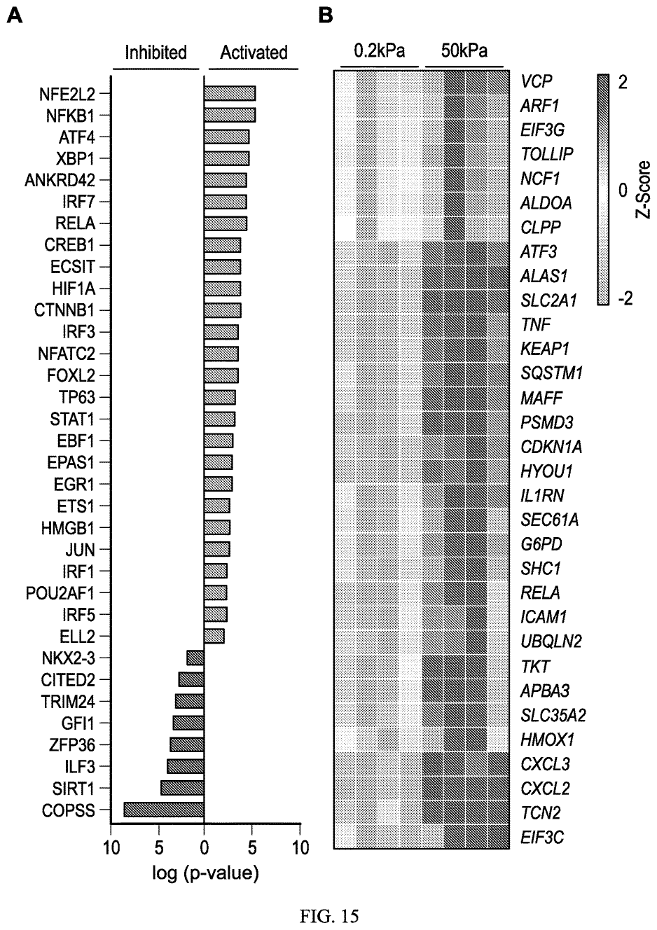

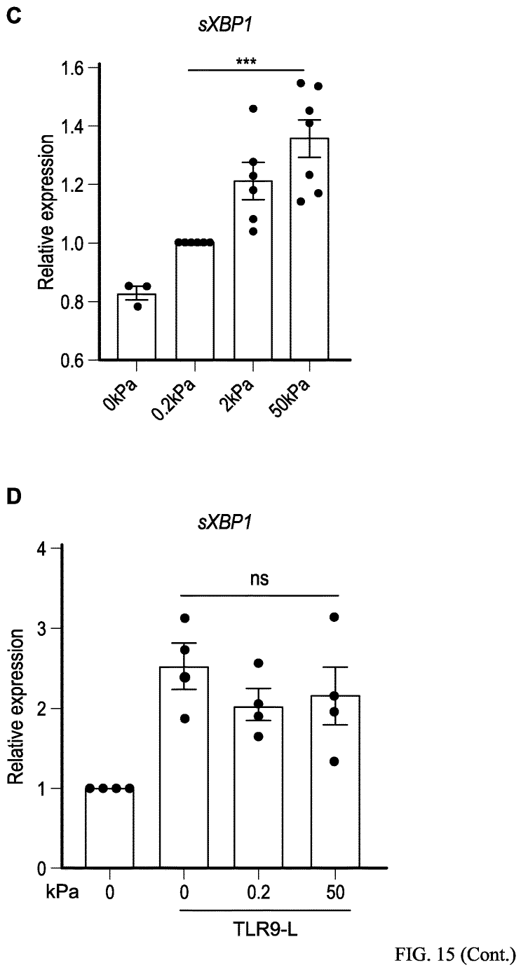

Despite pDCs expressing classical mechanotransduction-associated genes, including genes involved in cell movement, in response to a stiff microenvironment (FIGS. 7B and 7C), our analysis unveiled a notable absence of key genes involved in the Hippo pathway such as TEAD1/2/3/4 and the YAP1 binding factor, while LATS1, LATS2 and TAZ were weakly expressed in human pDCs (FIG. 14A), suggesting that this classical pathway may not operate in pDCs. To further support this finding, we tested this functionally using SuperTDU131, a peptide which by binding to TEADs, prevents YAP1 binding (Jiao et al., 2017), and observed that blocking YAP1 did not rescue IFN-α production in pDCs at high stiffness (FIG. 14B). These data strongly suggest that stiffness signals pDCs without the involvement of the YAP1/TEADs pathway. To explore this further, pDCs with a TLR9 agonist under low (0.2 kPa) or high (50 kPa) stiffness conditions were cultured and an unbiased transcriptomic analysis was performed using RNA-seq. The principal component analysis (PCA) revealed a clear segregation of the two sets of conditions (FIG. 8A) associated with substantial downregulation of various IFN-I and IFN-III genes (FIG. 8B). The regulation of transcription factors was then tested, and as expected, there was a strong downregulation of IRF7 in TLR9-activated pDCs experiencing a stiff microenvironment (FIG. 8C). Strikingly, a significant activation of multiple stress was observed associated TFs along with NFE2L2 (NF-E2-related factor 2), also known as NRF2, being the highest induced gene under these conditions (FIG. 8C). Furthermore, NRF2 was also induced by high stiffness, independently of TLR9 signaling (FIG. 15A). Additional stress-associated TFs were induced, such as ATF4 and XBP1 (FIGS. 8C and 15B), which we recently showed to control pDC responses (Chaudhary et al., 2022), although TLR9 signaling seemed to trump the induction of sXBP1 (FIGS. 15C and 15D). Additionally, stiffness alone can induce other inflammatory related TFs such as NF-κB, IRF5 which fits the previously described observation in murine BMDM derived macrophages (Meli et al., 2020). NRF2 is a member of the Cap‘n’collar (CNC) transcription factor family and is responsible for regulating oxidative stress responses (Ma, 2013). Looking at differentially expressed genes, our analysis revealed genes associated with various pathways, including the induction of genes associated with cell migration (CXCL2, CXCL3), oxidative stress response (NQO1, HMOX1, SQSTM1) and the pentose phosphate pathway (TKT) and decreased interferon production (IFNB1) (FIGS. 8D and 15B). In the normal hemostatic state, NRF2 undergoes ubiquitination mediated by the KEAP1 protein, thus reducing NRF2 protein levels. However, when activated, ubiquitination is temporarily halted, resulting in increased NRF2 levels (Baird and Yamamoto, 2020; Ma, 2013). A specific activator of NRF2, called KI696 (Davies et al., 2016) which was confirmed could induce the expression of HMOX1, a classical NRF2-dependent gene (Ma, 2013) in pDCs (FIG. 8E). To determine the effect on NRF2 at the protein level, both intracellular flow cytometry as well as western Blot were used, as measuring protein level is a good surrogate to predict nuclear translocation (Mills et al., 2018). The data show that indeed, NRF2 is induced by high stiffness in pDCs, with KI696 as a positive control (FIGS. 8F-H). High stiffness also induced the expression of HMOX1 in pDCs (FIG. 8I).

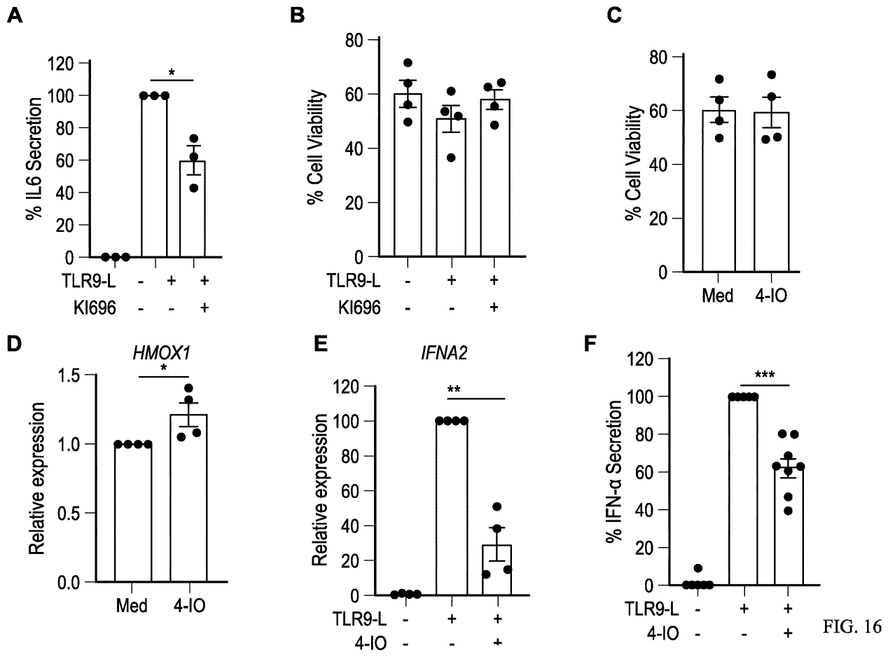

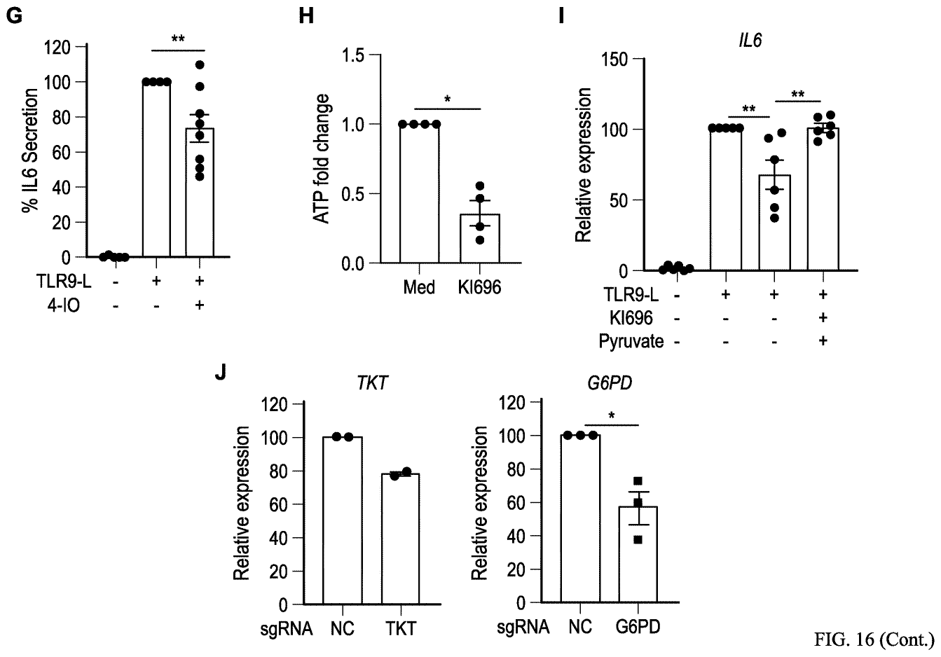

To determine the functional consequence of NRF2 activation, cells were activated with a TLR9 agonist, CpG-C274, in the presence of KI696. The data show that the activation of NRF2 significantly inhibited expression and secretion of IFN-α in TLR9-activated pDCs (FIGS. 8J and K) along with IL6 secretion (FIG. 16A) without affecting cell viability (FIG. 16B). This latter observation was confirmed using a second NRF2 activator, 4-Octyl Itaconate (Mills et al., 2018), with similar effect on the induction of HMOX1 and inhibition of IFN-α and IL-6 without impacting cell viability (FIGS. 16C-G). Upon activation, NRF2 undergoes nuclear translocation, where it forms a complex with the s-Maf protein, enabling its binding to gene promoters to initiate transcription (Baird and Yamamoto, 2020; Ma, 2013). To further validate the direct role of NRF2 in mediating stiffness-induced signaling, we employed an inhibitor specifically designed to prevent the binding of s-Maf with NRF2 (Singh et al., 2016). The data show that blocking NRF2 activation in pDCs under high stiffness conditions rescued the expression of IFNA (FIG. 8L). These findings indicate that mechanosensing in pDCs promotes transcriptional activity of NRF2, which serves as a key regulator of TLR9 signaling and IFN-I production in pDCs.

### Example 9: High Stiffness Induces the Pentose Phosphate Pathway which Reduces Glycolysis that is Needed for IFN-I Response in pDCs

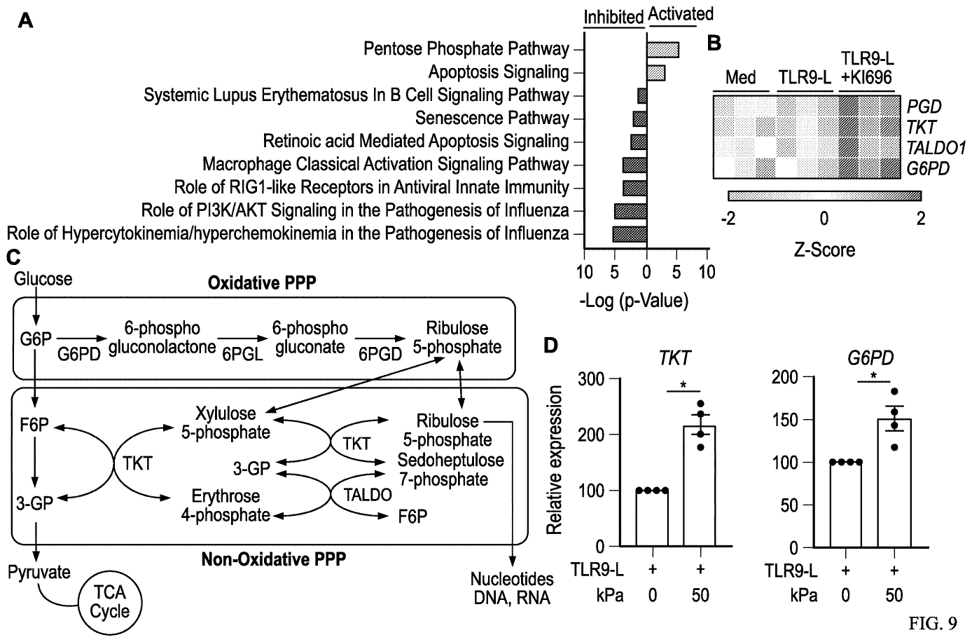

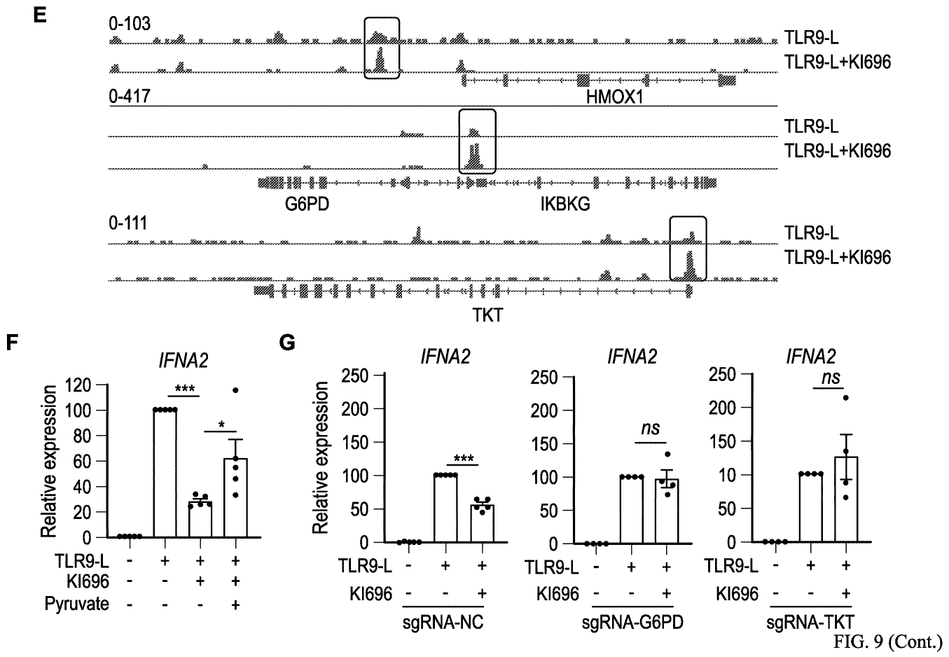

To investigate how transcriptional activation of NRF2 inhibits IFN-I, a transcriptomic analysis of pDCs activated with KI696 was performed, and showed the involvement of the pentose phosphate pathway (PPP) (FIG. 9A). The activation of NRF2 in TLR9-activated pDCs led to a significant induction of genes involved in PPP pathways such as Glucose 6 phosphate dehydrogenase (G6PD), Transketolase (TKT), Phosphogluconate Dehydrogenase (PGD) and transaldolase 1(TALDO1) (FIG. 9B). The PPP is interconnected with glycolysis through two key enzymes: G6PD acting on glucose 6-phosphate (G6P) and the Transketolase (TKT) on Fructose 6-phosphate (F6P) and glyceraldehyde 3-phosphate (3-PG) (Patra and Hay, 2014; Stincone et al., 2015) (FIG. 3C). G6PD is the rate-limiting enzyme of PPP that breaks down glucose to generate ribulose 5-phosphate, a precursor of DNA, RNA, and ATP, with the concomitant generation of NADPH molecules (Gomez-Manzo et al., 2016). Consequently, the expression of G6PD and TKT under increasing stiffness was examined, and confirmed their induction in pDCs experiencing elevated stiffness, regardless of TLR9 activation (FIG. 9D and FIG. 15B). To gain deeper insights into the link between the activation of NRF2 and transcriptional regulation of the PPP, a CUT&RUN assay was conducted to examine the binding of NRF2 to the promoter of the genes involved in this pathway. The binding of NRF2 on the promoter of HMOX1 and PPP genes such as G6PD and TKT (FIG. 9E) was observed.

The activation of the PPP can divert glycolysis away from pyruvate synthesis, leading to a deficiency in pyruvate levels within the cells (FIG. 9C). Recently, we have reported that pyruvate and the subsequent production of ATP are crucial for an IFN-I response in pDCs (Chaudhary et al., 2022). Indeed, it was observed here that ATP levels were reduced in pDCs treated with KI696 (FIG. 16H), and supplementing exogenous pyruvate could restore IFNA and IL6 expression in TLR9-activated pDCs in the presence of the activator of NRF2 (FIG. 9F and FIG. 16I). To provide further evidence of the direct involvement of G6PD in stiffness-mediated inhibition of IFN-I, we utilized the CRISPR-Cas9 technique was utilized to knock down this gene (FIG. 16J). Remarkably, it was observed that even the partial reduction in the expression of G6PD through the CRISPR-Cas9 system resulted in the restoration of IFNA2 expression in pDCs incubated with KI696 (FIG. 9G). A similar approach was then used with a comparable outcome when TKT was inhibited with the rescuing of IFN-I in pDCs (FIG. 9G and FIG. 16J). These results indicate that mechanosensing-driven activation of NRF2 induces PPP related genes which divert the proper glycolysis pathway which is necessary for optimal IFN-α responses in TLR9-activated pDCs.

### Example 10: Skin Stiffness Induces NRF2 Activation in Skin-Infiltrating pDCs to Allow Resolution of Inflammation by Inhibiting IFN Responses

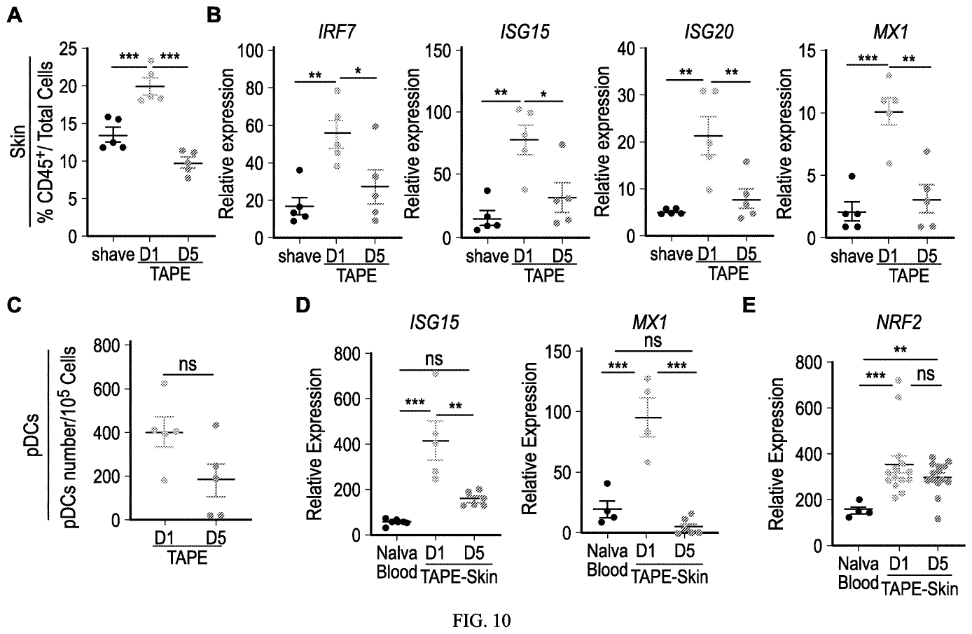

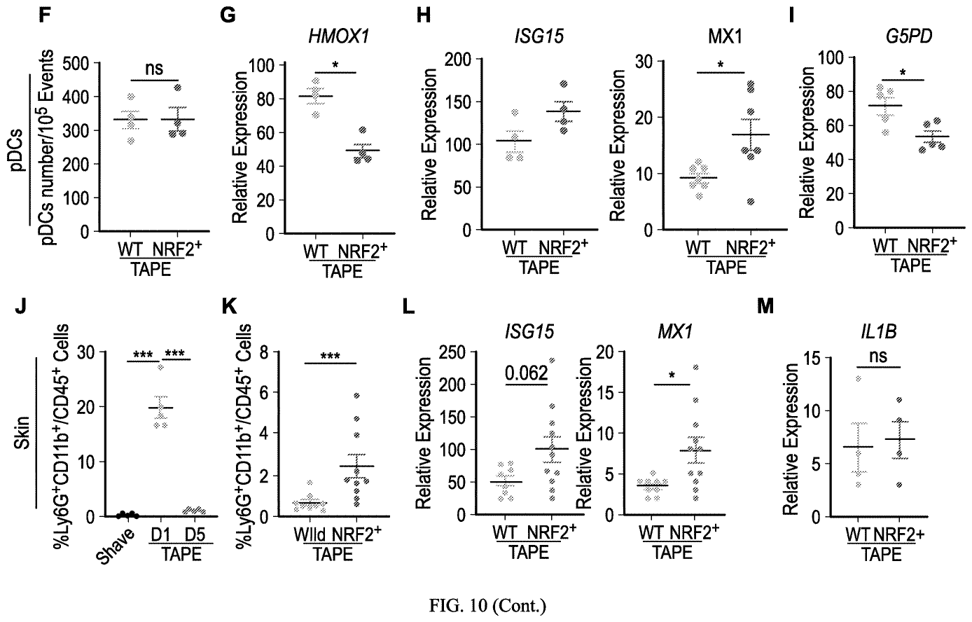

To determine the relevance of these findings in vivo, a mild skin injury inflammatory model by tape stripping was employed, which we and others have shown is associated with the induction of an IFN-I response due to the activation of pDCs in the skin (Ah Kioon et al., 2021; Du et al., 2022; Gregorio et al., 2010; Guiducci et al., 2010). Similar to previous findings, this acute model of skin injury induced a peak of inflammation at day 1, with significant infiltration of CD45+ cells in the skin, followed by resolution and tissue repair by day 5 (FIG. 10A). Consistent with this, the expression of ISGs, like ISG15, ISG20, MX1, and IRF7, followed a similar pattern in the inflamed skin (FIG. 10B). We then focused on pDCs and observed a similar increase in pDC infiltration into the skin by tape stripping on day 1 which at day 5 appeared reduced (FIG. 10C), while as reported, no pDCs can be detected in naïve skin. Hence this model allows the rapid infiltration of pDCs in the skin in a controlled manner, which changes the stiffness upon pDCs from 0.1 kPa (blood) to about 2 kPa (skin), a 200-fold increase. Considering that pDCs are known to detect skin injuries and induce ISG expression (Gregorio et al., 2010; Guiducci et al., 2010; Ross et al., 2021), we FACS-sorted pDCs from the blood and skin at the early and late time points after injury. First, the results showed that, in line with the data in total skin (FIG. 10B), the expression of ISGs by the pDCs themselves was consistent with a significant induction in skin-infiltrating pDCs at day 1 and resolution at day 5 (FIG. 10D). Strikingly, the data showed that NRF2 was induced in the pDCs that infiltrate the skin of the mice, as compared to blood, and that its expression did not decrease at day 5 (FIG. 10E). To determine the link between NRF2 activation and the downregulation of ISG expression in the skin at the later time point post-injury, tape stripping was performed using mice lacking NRF2 (NRF2-KO) and evaluated the inflammatory response at day 5. There is not an available strategy to genetically delete genes in pDCs, so we used total knockout mice, but did evaluate the effect specifically on pDCs by sorting the cells from the skin of the mice. Although there was little to no difference in the number of pDCs infiltrating the skin in NRF2-KO mice as compared to wildtype mice (FIG. 10F), the data showed that the expression of the NRF2-dependent gene HMOX1 was significantly downregulated in sorted pDCs from skin in the NRF2-ko mice (FIG. 10G). This demonstrated the engagement of NRF2 in pDCs once they infiltrate the skin. The impact of the deletion of NRF2 on the inflammatory response was profound, with increased expression of the IFN-regulated genes ISG15 and MX1 in sorted pDCs (FIG. 10H) which was associated with reduction of expression of G6PD in the skin-infiltrating pDCs (FIG. 10I), suggesting that the PPP is indeed regulated by NRF2 in pDCs once in the skin. The impact of NRF2 deletion was also evident in total skin with an increase in the number of neutrophils that infiltrate the skin of the NRF2-KO mice at day 5 as compared to wild-type mice (FIGS. 10J and K). This led to increased expression of ISGs in the skin (FIG. 10L), while we noted that there was no change in the expression of non-ISGs genes such as IL1B (FIG. 10M). These findings highlight that the activation of NRF2 in pDCs is associated with the inhibition of ISG in vivo and that in the absence of NRF2, there is a defect in the resolution of skin inflammation.

### Example 11: Activation of NRF2 Orchestrates the Switch from Inflammation to Resolution by Inhibiting Infiltrating pDCs

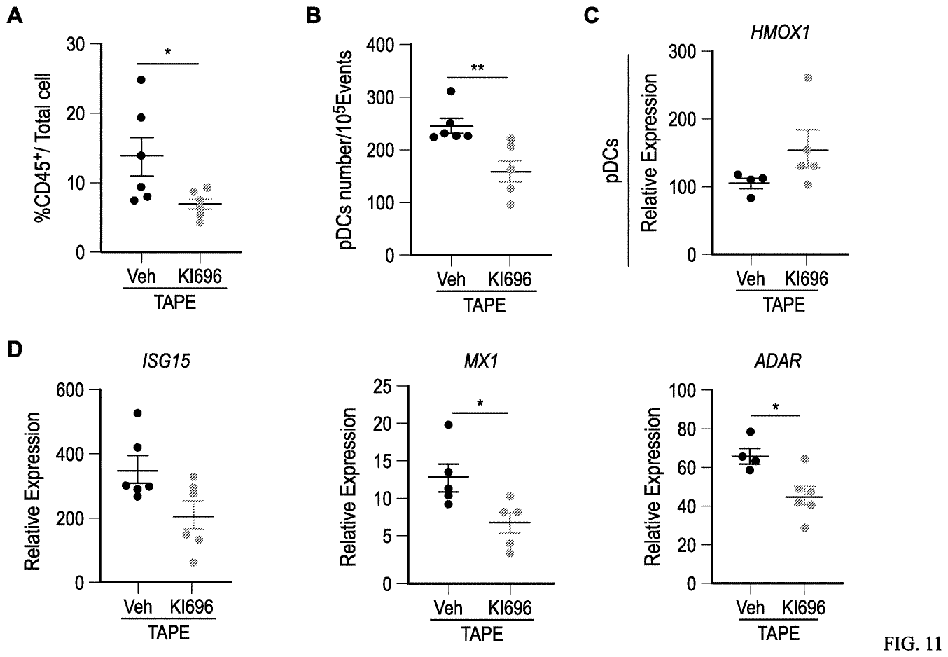

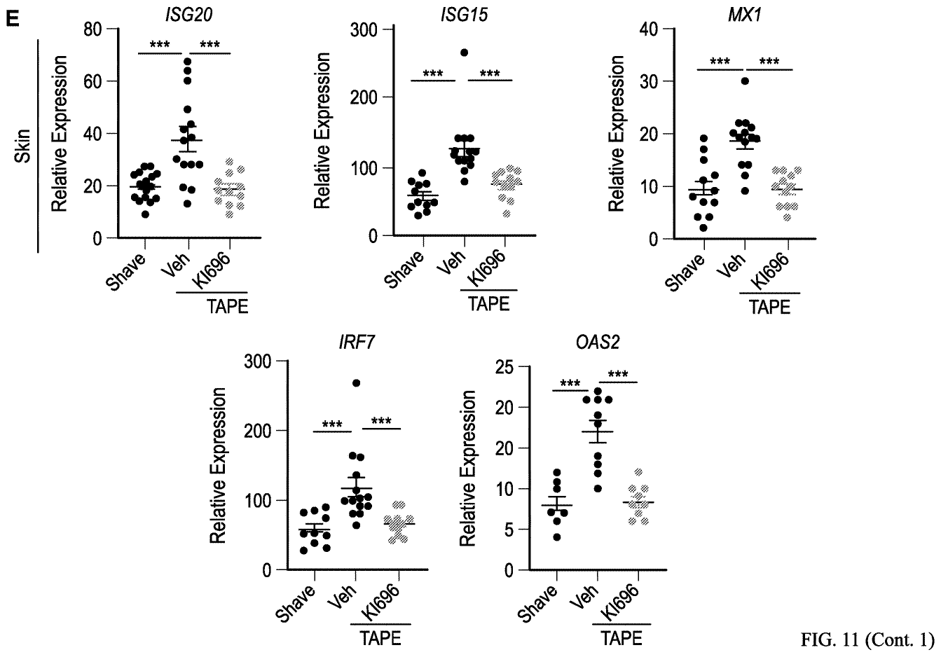

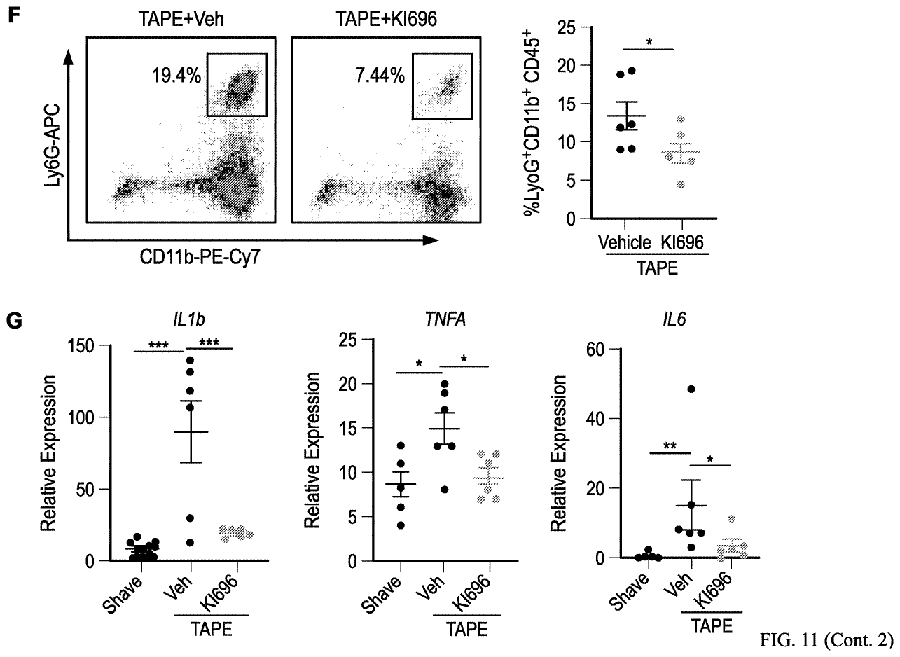

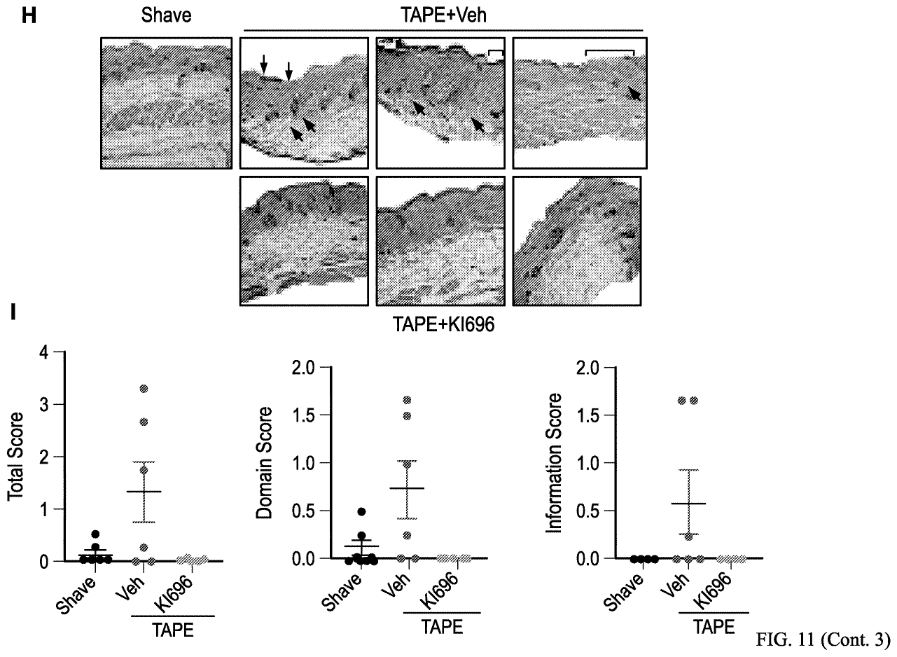

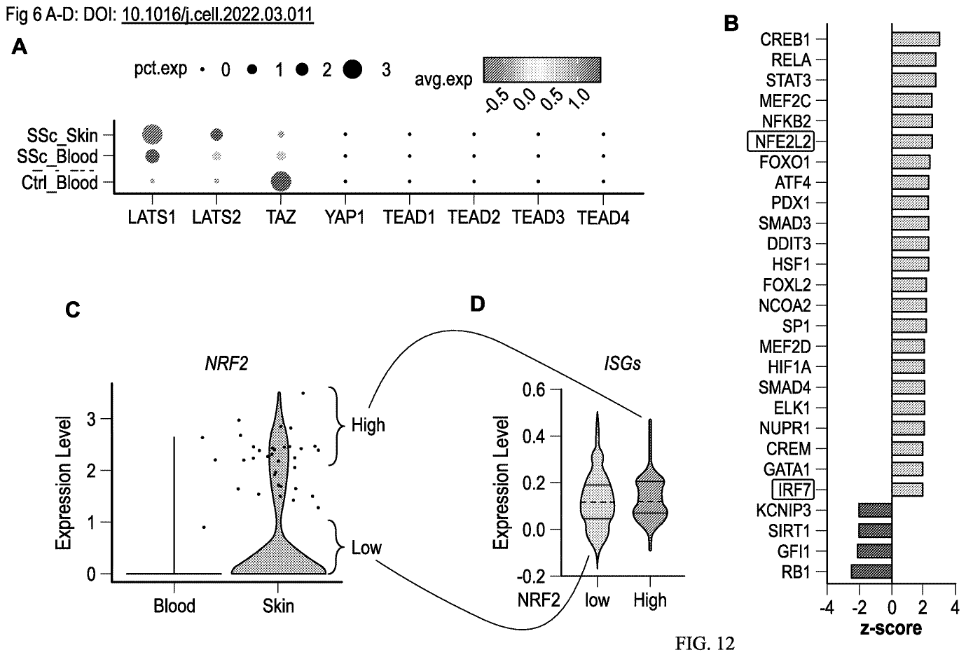

The data has shown that the modulation of NRF2 using the activator KI696 effectively inhibited IFN-I production in pDCs (FIGS. 8J and K). Skin injury was induced by tape stripping on wildtype mice, administering either a vehicle or KI696 via intraperitoneal injection using previously described dose for in vivo experiment (Davies et al., 2016). The impact of activating NRF2 had a significant effect on the inflammatory response in the skin. A decrease in the infiltration of CD45+ cells and of pDCs into the skin at day 1 following tape stripping was observed (FIGS. 11A and B). In order to determine the effect on pDCs, the pDCs were FACS-sorted from the skin and first, it was observed that KI696 induced HMOX1 expression in the skin-infiltrating pDCs (FIG. 11C), confirming that, although the treatment was given systemically, KI696 did indeed activate NRF2 in the pDCs in vivo. The activation of NRF2 in vivo led to reduced ISGs in purified pDCs (FIG. 11D) as well as in total skin (FIG. 11E). Additionally, we noted a reduction in neutrophil infiltration (FIG. 11F) and decreased levels of inflammatory cytokines such as IL6, IL1B, IL1A, and TNFA in the inflamed skin (FIG. 11G). Strikingly, the activation of NRF2 had an impact on the skin at the histological level with reduction in ulceration and inflammation in the KI696-treated mice (FIGS. 11H and I). Methods were used to determine skin barrier disruption, ulceration and inflammation score which have limitations but have been validated in previous studies (Ah Kioon et al., 2021; Ah Kioon et al., 2018; Guiducci et al., 2010). Hence, these data support a key role for NRF2 in regulating the IFN-I response in the skin and supports the use of an NRF2 activator to prevent or resolve skin lesions.

### Example 12: Dysregulated NRF2-Mediated pDC Response in the Skin of SSc Patients Exacerbates the IFN-I Response, Contributing to Fibrosis

To determine the role of NRF2 in controlling the pDC response in the skin in the human setting and to understand why ISGs are chronically expressed in the skin of autoimmune patients, a re-analysis of published sc-RNA-seq data performed on the blood and skin of healthy donors (n=56) and SSc patients (n=97) (DOI: 10.1016/j.cell.2022.03.011 (Gur et al., 2022) was conducted. In this work, the authors had reported the presence of pDCs in the skin of patients but not healthy volunteers (Gur et al., 2022), supporting our previous findings (Ah Kioon et al., 2018).

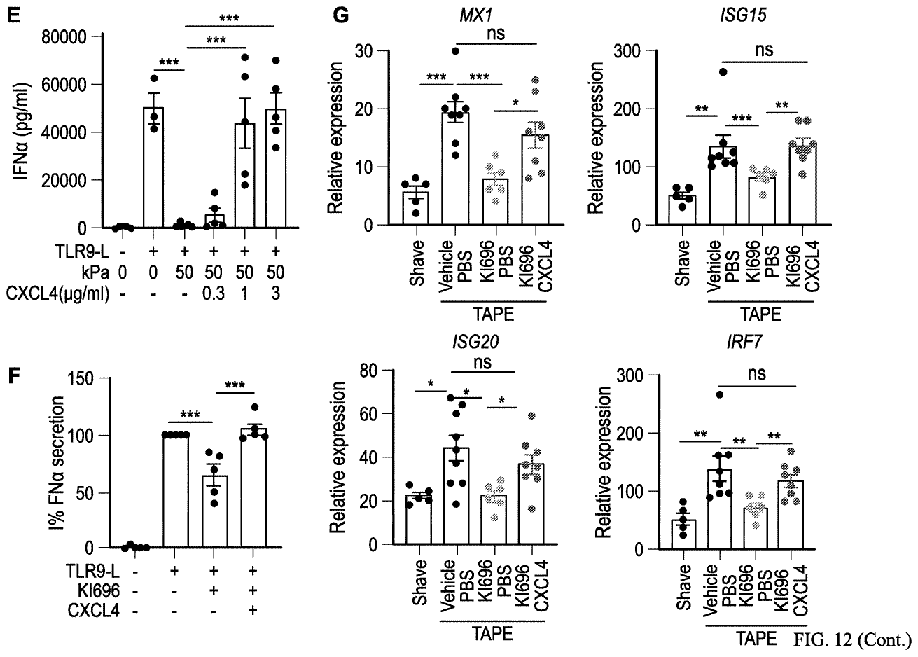

First, an absence of expression of YAP1 and TEAD1/2/3/4 in the pDCs that infiltrated the skin (FIG. 12A) was confirmed in patients. Next, we assessed the impact of stiffness in pDCs from the blood and skin of SSc by performing a differential gene expression analysis and focused on TFs. We observed the activation of NRF2 in the skin-infiltrating pDCs, as compared to the blood of these patients (FIG. 12B), confirming the induction of NRF2 in skin-infiltrating pDCs. As previously reported by us and others, no pDCs were identified in healthy skin, so it was impossible to compare the expression levels of NRF2 in pDCs in the skin between SSc and HDs. A striking observation was the presence of IRF7 activation in these skin-infiltrating pDCs (FIG. 12B) suggesting that, in contrast to our observation in vitro using cells from HDs or in vivo in normal mice, the activation of NRF2 is uncoupled from the inhibition of IFN-I in pDCs infiltrating the skin of SSc patients. Indeed, pDCs with high versus low expression of NRF2 were separated (FIG. 12C) and no difference in the expression of ISGs between each group (FIG. 12D) was observed. This demonstrates that the inhibition of the IFN-I response by NRF2 in pDCs observed in normal settings does not occur in patients. We and others have reported that pDCs from patients with SSc exhibit sustained activation, resulting in continuous secretion of CXCL4 and promotion of IFN-α production (Ah Kioon et al., 2018; Du et al., 2022; Lande et al., 2019; van Bon et al., 2014). The role of CXCL4, which is elevated in patients with SSc (van Bon et al., 2014; Volkmann et al., 2016), in promoting pDC activation has been well documented, and we have shown that CXCL4 can be induced in inflamed and fibrotic skin leading to increased IFN-I by skin-infiltrating pDCs (Ah Kioon et al., 2018; Du et al., 2022). Hence, it was tested here whether CXCL4 could hinder the regulatory effect of stiffness-induced NRF2 in pDCs. Strikingly, the addition of CXCL4 could totally overcome the inhibitory effect of high stiffness on IFN-α production by pDCs (FIG. 12E) was observed. This effect was also observed when cells were incubated with KI696 to provoke NRF2-mediated inhibition (FIG. 12F). These findings demonstrate that CXCL4 can overcome the inhibitory effects of stiffness and NRF2 activation in pDC. To experimentally validate these findings in vivo, CXCL4 was administered to the inflamed skin of tape-stripped mice along with an NRF2 activator KI696. As previously observed, tape stripping induced ISGs in the skin of the mice, and the data showed that the induction of the ISGs was inhibited by KI696 (FIG. 12G). The addition of CXCL4 completely reverted this inhibition, with the engagement of NRF2 no longer leading to the inhibition of IFN-I in the skin (FIG. 12G).

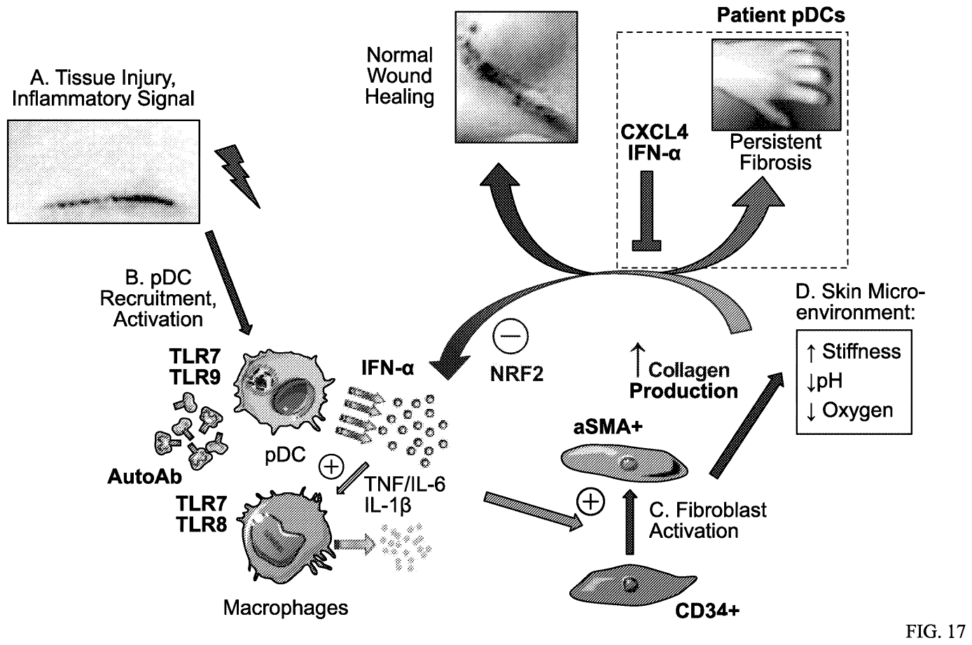

In summary, these data suggest that when reaching the skin, pDCs can sense nucleic acids and become activated, inducing a strong IFN-I response, but the stiffness of the skin microenvironment and the resulting mechanosensing of integrin-mediated signals induce a sustained expression of NRF2 which promotes the resolution of the inflammatory response. In contrast to that healthy response, in patients with autoimmune diseases, the presence in the skin of CXCL4 overcome this normal wound healing process to allow the chronic activation of the pDCs and establishment of fibrosis (FIG. 17).

Various modifications of the invention, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

1. A method of inhibiting pDC activity in skin tissue, said method comprising activation of NRF2.

2. (canceled)

3. The method of claim 1, wherein the pDC is activated by TLR7 and/or TLR9.

4. The method of claim 1, wherein the activation of NRF2 is by exposure to an NRF2 activator.

5. The method of claim 4, wherein the NRF2 activator is KI696 or 4-octyl itaconate.

6. (canceled)

7. The method of claim 1, wherein activating NRF2 induces the Pentose Phosphate pathway.

8. A method of inhibiting pDC activity in skin tissue, said method comprising inducing the Pentose Phosphate pathway.

9. (canceled)

10. The method of claim 8, wherein the pDC is activated by TLR7 and/or TLR9.

11. The method of claim 8, wherein the Pentose Phosphate pathway is induced by G6PD induction or activation, or by TKT induction or activation.

12. The method of claim 11, wherein the G6PD is induced or activated by exposure to a G6PD inducer.

13. (canceled)

14. The method of claim 11, wherein the TKT is induced or activated by exposure to a TKT inducer.

15. The method of any one of claim 1, wherein inhibiting the pDC activity inhibits IFN secretion.

16. A method of treating a disease or disorder associated with inhibition of pDC activity, said method comprising activation of NRF2.

17. The method of claim 16, wherein the activation of NRF2 is by administration of an NRF2 activator.

18. The method of claim 17, wherein the NRF2 activator is KI696 or 4-octyl itaconate.

19. (canceled)

20. A method of treating a disease or disorder associated with inhibition of pDC activity, said method comprising inducing the Pentose Phosphate pathway.

21. The method of claim 20, wherein the Pentose Phosphate pathway is induced by G6PD induction or activation, or by TKT induction or activation.

22. The method of claim 21, wherein the G6PD is induced or activated by administration of a G6PD inducer.

23. (canceled)

24. The method of claim 21, wherein the TKT is induced or activated by administration of a TKT inducer.

25. The method of claim 16, wherein the disease or disorder is selected from the group consisting of an autoimmune disorder, a fibrotic disorder, a skin disease or disorder, fibrosis, and systemic lupus erythematosus (SLE).

26.-28. (canceled)

29. The method of claim 25, wherein the skin disease or disorder is a member selected from the group consisting of systemic sclerosis, psoriasis, scleroderma, cutaneous lupus, acute cutaneous lupus (ACLE), chronic cutaneous lupus (discoid lupus), cutaneous lupus erythematosus (CLE), dermatomyositis, Behcet's Disease, ocular cicatricial pemphigoid (OCP), pemphigus, epidermolysis bullosa, epidermolysis bullosa acquisita (EBA), bullous pemphigoid, lichen planus, and lichen sclerosus.

30.-44. (canceled)


SEQUENCE LISTING (36 sequences)

SEQ ID NO: 1 - DNA, 19 bp, Homo sapiens
  source 1..19: mol_type = other DNA
cacttggtcctgcgcttga

SEQ ID NO: 2 - DNA, 24 bp, Homo sapiens
  source 1..24: mol_type = other DNA
caattgggaatgcaacaactttat

SEQ ID NO: 3 - DNA, 20 bp, Homo sapiens
  source 1..20: mol_type = other DNA
tacccccaggagaagattcc

SEQ ID NO: 4 - DNA, 20 bp, Homo sapiens
  source 1..20: mol_type = other DNA
gccatctttggaaggttcag

SEQ ID NO: 5 - DNA, 19 bp, Homo sapiens
  source 1..19: mol_type = other DNA
cccaggaggagtttggcaa

SEQ ID NO: 6 - DNA, 21 bp, Homo sapiens
  source 1..21: mol_type = other DNA
tgctggatcatctcatggagg

SEQ ID NO: 7 - DNA, 19 bp, Homo sapiens
  source 1..19: mol_type = other DNA
ttcccggtcacatcgagag

SEQ ID NO: 8 - DNA, 23 bp, Homo sapiens
  source 1..23: mol_type = other DNA
tcctgttgcataccgtctaaatc

SEQ ID NO: 9 - DNA, 21 bp, Homo sapiens
  source 1..21: mol_type = other DNA
aaagccctacagcaactgtcg

SEQ ID NO: 10 - DNA, 21 bp, Homo sapiens
  source 1..21: mol_type = other DNA
aagactgcgttcctgctcaac

SEQ ID NO: 11 - DNA, 21 bp, Homo sapiens
  source 1..21: mol_type = other DNA
aacatcgcctgcgttatcctc

SEQ ID NO: 12 - DNA, 19 bp, Homo sapiens
  source 1..19: mol_type = other DNA
acgtcccggatgatcccaa

SEQ ID NO: 13 - DNA, 21 bp, Homo sapiens
  source 1..21: mol_type = other DNA
tcatcgagtgctacattgctg

SEQ ID NO: 14 - DNA, 21 bp, Homo sapiens
  source 1..21: mol_type = other DNA
gccatgcgaatctggtcaaag

SEQ ID NO: 15 - DNA, 22 bp, Mus musculus
  source 1..22: mol_type = other DNA
tagatgaccatgagtcgcttgc

SEQ ID NO: 16 - DNA, 20 bp, Mus musculus
  source 1..20: mol_type = other DNA
gccaaacttgctccatgtcc

SEQ ID NO: 17 - DNA, 21 bp, Mus musculus
  source 1..21: mol_type = other DNA
aagccgagaatgctgagttca

SEQ ID NO: 18 - DNA, 23 bp, Mus musculus
  source 1..23: mol_type = other DNA
gccgtgtagatatggtacaagga

SEQ ID NO: 19 - DNA, 20 bp, Mus musculus
  source 1..20: mol_type = other DNA
aaatgatgccttccaccaag

SEQ ID NO: 20 - DNA, 20 bp, Mus musculus
  source 1..20: mol_type = other DNA
gcatagcccacaatgaaggt

SEQ ID NO: 21 - DNA, 23 bp, Mus musculus
  source 1..23: mol_type = other DNA
atggaaggttaccataagccaga

SEQ ID NO: 22 - DNA, 19 bp, Mus musculus
  source 1..19: mol_type = other DNA
tgcagcatgatgtggggtg

SEQ ID NO: 23 - DNA, 21 bp, Mus musculus
  source 1..21: mol_type = other DNA
acggtcttaccctttccagtc

SEQ ID NO: 24 - DNA, 20 bp, Mus musculus
  source 1..20: mol_type = other DNA
cccctttcgttcctcaccag

SEQ ID NO: 25 - DNA, 20 bp, Mus musculus
  source 1..20: mol_type = other DNA
gtcacgcctcagcacatggt

SEQ ID NO: 26 - DNA, 21 bp, Mus musculus
  source 1..21: mol_type = other DNA
ccaccagcttgcctttcagaa

SEQ ID NO: 27 - DNA, 20 bp, Mus musculus
  source 1..20: mol_type = other DNA
tctgtgcaggcactatgagg

SEQ ID NO: 28 - DNA, 20 bp, Mus musculus
  source 1..20: mol_type = other DNA
gcctctccactcctctcctt

SEQ ID NO: 29 - DNA, 20 bp, Mus musculus
  source 1..20: mol_type = other DNA
acagggcgttttatcttgcg

SEQ ID NO: 30 - DNA, 19 bp, Mus musculus
  source 1..19: mol_type = other DNA
tccaagctcccggctaagt

SEQ ID NO: 31 - DNA, 25 bp, Mus musculus
  source 1..25: mol_type = other DNA
aaaccgtttttccatcttcttcttt

SEQ ID NO: 32 - DNA, 17 bp, Mus musculus
  source 1..17: mol_type = other DNA
gacggcacacccaccct

SEQ ID NO: 33 - DNA, 24 bp, Mus musculus
  source 1..24: mol_type = other DNA
gcaagtggctagagtgcagagtaa

SEQ ID NO: 34 - DNA, 25 bp, Mus musculus
  source 1..25: mol_type = other DNA
tggctattaattattcggtctgcat

SEQ ID NO: 35 - DNA, 23 bp, Homo sapiens
  source 1..23: mol_type = other DNA
acgggcatagcccacgatgaagg

SEQ ID NO: 36 - DNA, 23 bp, Homo sapiens
  source 1..23: mol_type = other DNA
gaccgggtgcccgtccaagtcgg